SEQUENCE LISTING (17 sequences)

SEQ ID NO: 1 - DNA, 20 bp, synthetic construct
  source 1..20: mol_type = other DNA
tctggaacgtgagcataccc

SEQ ID NO: 2 - DNA, 20 bp, synthetic construct
  source 1..20: mol_type = other DNA
cggctcaaagaccttgctca

SEQ ID NO: 3 - DNA, 20 bp, synthetic construct
  source 1..20: mol_type = other DNA
gctaccacaggttccagtcc

SEQ ID NO: 4 - DNA, 21 bp, synthetic construct
  source 1..21: mol_type = other DNA
cgacggttataactgctggtg

SEQ ID NO: 5 - DNA, 19 bp, synthetic construct
  source 1..19: mol_type = other DNA
ccagtggccaacctgagtg

SEQ ID NO: 6 - DNA, 19 bp, synthetic construct
  source 1..19: mol_type = other DNA
tggcggctgtgtctgttag

SEQ ID NO: 7 - DNA, 20 bp, synthetic construct
  source 1..20: mol_type = other DNA
caagagtgcgtctcggacag

SEQ ID NO: 8 - DNA, 20 bp, synthetic construct
  source 1..20: mol_type = other DNA
ttcatctggccaggacactg

SEQ ID NO: 9 - DNA, 25 bp, synthetic construct
  source 1..25: mol_type = other DNA
caccgctctacgatcttctgccggg

SEQ ID NO: 10 - DNA, 25 bp, synthetic construct
  source 1..25: mol_type = other DNA
aaaccccggcagaagatcgtagagc

SEQ ID NO: 11 - DNA, 20 bp, synthetic construct
  source 1..20: mol_type = other DNA
tccagtgggcaggttcaaat

SEQ ID NO: 12 - DNA, 22 bp, synthetic construct
  source 1..22: mol_type = other DNA
aggacgcttagagtggagggcc

SEQ ID NO: 13 - DNA, 20 bp, synthetic construct
  source 1..20: mol_type = other DNA
atcaacttcgactggccctt

SEQ ID NO: 14 - DNA, 20 bp, synthetic construct
  source 1..20: mol_type = other DNA
ccgtacatgtcgatgttcac

SEQ ID NO: 15 - DNA, 20 bp, synthetic construct
  source 1..20: mol_type = other DNA
ttccccatggcncacaayac

SEQ ID NO: 16 - DNA, 23 bp, synthetic construct
  source 1..23: mol_type = other DNA
tgcckrctcatrggctgraagtt

SEQ ID NO: 17 - DNA, 32 bp, Mus musculus
  source 1..32: mol_type = genomic DNA
tgccggactccacccggcagatcgtagagcta

(19) United States
(12) Patent Application Publication
(10) Pub. No.: US 2026/0265687 A1
(43) Pub. Date: Sep. 10, 2026

(54) OCULAR ORGANOIDS

(71) Applicant: KONINKLIJKE NEDERLANDSE AKADEMIE VAN WETENSCHAPPEN, UTRECHT (NL)

(72) Inventors: Johannes Carolus CLEVERS, UTRECHT (NL); Marie BANNIER HÉLAOUËT, UTRECHT (NL); Amanda ANDERSSON-ROLF, UTRECHT (NL); Martina CELOTTI, UTRECHT (NL)

(21) Appl. No.: 19/167,257

(22) PCT Filed: Mar. 21, 2024
(86) PCT No.: PCT/NL2024/050143

§ 371 Date: Sep. 19, 2025

(30) Foreign Application Priority Data
Mar. 21, 2023 (NL) 2034392

(51) Int. Cl. C12N 5/079; A61K 35/30

(52) U.S. Cl. CPC C12N 5/0621; A61K 35/30; C12N 2501/15; C12N 2501/155; C12N 2501/415

(57) ABSTRACT

The invention relates to an in vitro method for the production of conjunctiva or limbal organoids. It also encompasses new conjunctiva or limbal organoids and their uses as implants in regenerative medicine, and as conjunctiva model or corneal limbus tissue and/or corneal tissue model for the study of this tissue. Particular cell culture media are also disclosed, which are in particular suitable for the production of the conjunctiva organoids or limbal organoids.

### FIELD OF THE INVENTION

This invention pertains in general to the field of organoids and methods for their production. It also relates to particular uses of the organoids in the therapeutic field and in the field of compound screening.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

The conjunctiva lines the inner surface of the eyelid and also covers the sclera (white of the eye). It is described to consist of a stromal layer, called substantia propria, containing fibroblasts, blood vessels and immune cells, which is covered by a non-keratinizing stratified epithelium.

Two differentiated cell types have been discerned in the epithelium: mucus-producing Goblet cells and keratinocytes. Most studies on the conjunctiva have focused on Goblet cells since these-together with the tear gland-produce the mucin layer of the tear film that covers the ocular surface. This mucin layer is essential for tear film homeostasis as it allows the aqueous layer of the tears to adhere to the ocular surface and protects against evaporative dry eye disease. However, neither a role of conjunctival keratinocytes beyond creating an epithelial barrier nor the identity of the conjunctival stem-cell has been discerned.

Studies on the ocular surface have primarily focused on one tissue essential for vision, the cornea. Nevertheless, the conjunctiva is equally essential for sight: its malfunction eventually affects corneal homeostasis and can lead to blindness. First, the avascular cornea is an immune-privileged tissue protected from extensive immune responses. It is the conjunctiva that provides immune protection to the ocular surface. Second, if physical or viral insults damage the conjunctiva, the tear film is destabilized, leading to dry eye disease, discomfort, and eventually blindness.

The corneal limbus is the border between the cornea and the conjunctiva (covering the sclera, i.e. the white of the eye) and contains limbal stem-cells. The cornea constitutes the transparent layer of the anterior eye. This layer consists of extracellular matrix-secreting keratocytes organized perpendicularly to light trajectory covered by a non-keratinized stratified epithelium. This epithelium is renewed by stem-cells located around the cornea, in a region called the limbus. During homeostasis and after injury of the central cornea, these stem-cells divide and migrate centripetally to differentiate and replenish the tissue. If an injury occurs in the limbus area, this can lead to limbal stem-cell deficiency. In such a case, the limbal stem-cells, and ultimately the central cornea, become replaced by non-transparent conjunctival tissue leading to blindness.

Because of this, both the cornea and the limbus have been the subject of intense investigations. Some culture models exist to study the cornea. These models are mostly either limbal stem-cells expanded on mouse embryonic fibroblasts (2D structure) or induced pluripotent stem-cells (iPSC)-derived organoids (3D structure).

The gold standard surgical procedure to restore a defect in the conjunctiva is autologous conjunctival graft. In brief, this means that a piece of healthy conjunctival tissue of the size of the damage (1 cm² on average) is directly taken from the patient's eye to cover the damage. This also means that the sclera where the healthy conjunctiva is taken is left bare, which makes it more prone to develop infections. Besides, if the damage is too large or in case of additional need for healthy conjunctival tissue, this option no longer stands. No ex vivo cell therapy for the conjunctiva is available to patients to date. Therefore, there is a clear unmet need in the clinic at the moment.

Simple limbal epithelial transplantation (SLET) is used to treat unilateral limbal stem-cell deficiency (LSCD). It involves harvesting healthy limbal stem-cells from the healthy eye for autologous implantation into the diseased eye to restore corneal transparency and prevent conjunctivalization.

A major hurdle to the understanding of conjunctival homeostasis and disorders and/or limbal homeostasis and disorders is the lack of a representative in vitro model. Indeed, models of the conjunctival epithelium established over the years hold several limitations: they are either short-lived explants cultured under poorly defined conditions (i.e. on feeder cells or amniotic membranes) or are derived from induced pluripotent stem-cells that typically do not recapitulate the cellular diversity and maturity of the conjunctival epithelium. This lack of a representative in vitro model of a mammal conjunctival epithelia has hindered the understanding of its biology and the development of medications for conjunctival disorders. The same is true for limbal homeostasis and disorders.

An example of an in vitro model of conjunctiva is disclosed in the document of Noni et al., “Generation of functional conjunctival epithelium, including goblet cells, from human iPSCs”, Cell Reports 34, 108715. Nomi et al. generate conjunctival epithelial lineage cells from human iPSCs that have been expanded to form a two-dimensional, eye-like organoid. EGF and KGF work in conjunction to promote, respectively, the development and maturation of the iPSC-derived conjunctival epithelium, which contains mucin-producing goblet cells. The model proposed by Nomi et al., does not reproduce the natural phenomena that takes place in the eye when conjunctiva is formed. In addition, this model needs a first step of forming a previous ectodermal autonomous multi-zone (SEAM), which makes the procedure to obtain the conjunctival epithelia a complex one.

Viral conjunctivitis is commonly studied using cancer cell lines derived e.g. from the lung. However, no protocol is available that allows the growth of primary conjunctival tissue long-term and under defined conditions.

An example of an in vitro model of human limbal (or corneal) epithelial cells, using human amniotic membrane (hAM) as the culture substrate has been described by Mariappan et al (Nat Protoc 2010 5 (8): 1470-9. doi: 10.1038/nprot.2010.115). Kauppila et al discuss further attempt to provide corneal limbus in vitro models (Advanced Healthcare Material 2023 Volume 12, Issue 29; doi.org/10.1002/adhm.202301396).

Adult stem-cell-based organoids were first established in 2009 from intestinal tissue. Organoids are three-dimensional structures that recapitulate essential architecture and functions of the tissue of origin, and they have been repeatedly proposed by regenerative medicine mainly of digestive organs, such as gut and liver. However, obtaining organoids that recapitulate key features of the tissue is not trivial. This is besides the fact that is also difficult to produce sufficient amounts of organoids to restore the organ, in particular the large ones. Hence, nowadays few clinical trials are ongoing to test the restorative capacities of organoids.//pct

In light of all this, new products, compositions, methods and uses would be highly desirable but are not yet readily available. In particular, there is a clear need in the art for reliable, efficient, and reproducible products, compositions, methods and uses that allow to be used in obtaining cell cultures, for example organoids, that recapitulate the essential structural and physiological features of actual conjunctiva epithelium or corneal epithelium and/or limbal epithelium and that may, for example, be used as transplant, for example in the treatment of cancer. Accordingly, the technical problem underlying the present invention can been seen in the provision of such products, compositions, methods and uses for complying with any of the aforementioned needs, or at least providing the public with a useful choice. The technical problem is solved by the embodiments characterized in the claims and herein below.

### SUMMARY OF THE INVENTION

As embodied and broadly described herein, the present invention is directed to the surprising finding that organoids of conjunctiva epithelium, corneal epithelium and/or limbal epithelium that recapitulate the essential structural and physiological features of actual conjunctiva epithelium or corneal epithelium and/or limbal epithelium, can be obtained by means of directly expanding and differentiating conjunctival stem-cells (for corneal epithelium), for example isolated from conjunctiva biopsies (comprising such conjunctival stem-cells) or by directly expanding and differentiating limbal stem-cells (for corneal epithelium and/or limbal epithelium, preferably for corneal epithelium or for limbal epithelium), for example isolated from corneal limbus biopsies or corneal biopsies (comprising such limbal stem-cells).

Preferably, the conjunctival stem-cells and/or the limbal stem-cells are obtained from primary tissue, for example from a (human) conjunctiva biopsy sample or from a (human) corneal limbus biopsy sample or (human) cornea biopsy sample.

Herewith is provided a reliable and simple in vitro method for the production of an organoid of the conjunctiva (using conjunctival stem-cells), and for the production of a limbal organoid (using limbal stem-cells), also referred herein to as a corneal limbal organoid, and which organoids avoid departing from iPSC and their associated disadvantages. The organoid of the conjunctiva (or conjunctiva organoid) can recapitulate essential structural and physiological features of actual conjunctiva epithelium. The limbal organoid can recapitulate essential structural and physiological features of actual corneal epithelium and/or limbal epithelium.

The new method disclosed herein allows to obtain enough amounts of conjunctiva organoids or limbal organoids in a reasonable and practical period of time, being thus useful for regenerative medicine, among other applications that will be disclosed.

The method uses simplified cell culture mediums that allow the proper expansion and/or differentiation of the conjunctiva stem-cells or of the limbal stem-cells. Surprisingly it was found that for conjunctiva stem-cells and for limbal stem-cells the same method of culturing, as disclosed herein, can advantageously be employed, allowing, for example, for long term culturing (for example more than 10, 20 or 30 passages) of the organoids, and providing, for example in ALI cultures, tissue structures closely resembling conjunctiva tissue, in particular conjunctiva epithelium, or limbal and/or cornea tissue, in particular limbal epithelium and/or corneal epithelium.

Furthermore, the organoids have an appropriate lifespan, which makes them useful as conjunctiva tissue models, or corneal limbus tissue and/or cornea tissue (in particular limbal epithelium and/or corneal epithelium). These models allow for the test of compounds that can modulate the conjunctiva epithelium or the limbal epithelium and/or corneal epithelium. The models can reliably reproduce pathological conditions of the conjunctiva or of the corneal limbus and/or cornea ((in particular limbal epithelium and/or corneal epithelium), reason for which they are also useful as models for the screening of drug candidates for the prevention and/or treatment of conjunctiva diseases or for the prevention and/or treatment of corneal limbus and/or cornea diseases.

Finally, also provided herewith are ready-to-use cell culture tools, reagents, and devices for the easy and reliable performance of the in vitro method of the invention.

Thus, in a first aspect the invention relates to an in vitro method for producing a conjunctiva organoid or a limbal organoid, comprising the steps of:
(a) providing conjunctival stem-cells or providing limbal stem-cells;
(b) culturing the conjunctival stem-cells or the limbal stem-cells in a culture medium, and under conditions suitable to form a conjunctiva organoid or to form a limbal organoid.

As will be understood by the skilled person, according to another aspect of the invention, conjunctival stem-cells and limbal stem-cells, both preferably obtained from a biopsy sample of tissue comprising such stem-cells, may be provided together in the same in vitro method for producing the organoids. It will therefore also be understood by the skilled person that the information disclosed herein relating to the method of culturing, including ALI culturing, the culture media to be used, the use of the thus obtained organoids or ALI cultures, and the organoids thus obtained, likewise, and in all embodiments apply to this aspect of the invention, and wherein the conjunctiva stem-cells and the limbal stem-cells are both introduced in the method of culturing. For example, it is contemplated that according to such aspect organoids may be obtained that have the characteristics on conjunctiva organoids as described herein and have the characteristics of the limbal organoids as described herein. The skilled person understand this without the need to verbally repeat description thereof.

As will illustrated in the examples below, these conjunctiva organoids comprise the basal cells and keratinocytes commonly found in actual conjunctiva epithelium. The cells are, moreover, spatially organized resembling actual architecture of this conjunctiva epithelium in vivo. This means that cells are disposed in stratified layers, comprising mainly the basal cells disposed at the bottom layers, in relation to a support or an extracellular matrix, or what in conjunctival tissue would be the substantia propria of the conjunctiva; and the differentiated keratinocytes are disposed on the top layers of the stratified structure, on the layers of basal cells. Moreover, the physiological features of the organoids also mimic those of the actual conjunctiva epithelium.

As will be illustrated in the examples below, the limbal organoids also comprise various cells, as witnessed from the expression of various cell markers, commonly found in actual corneal limbus tissue and/or corneal tissue (including corneal epithelium and/or limbal epithelium) and may display a spatial organization resembling that seen in corneal limbus tissue and/or corneal tissue (including corneal epithelium and/or limbal epithelium) in vivo.

With the method of the invention, sufficient amounts of organoids are obtained in a relatively short time. Moreover, this enough amount allows for the covering of an adequate surface area to be replaced or filled in a damaged or diseased conjunctiva or damaged or diseased corneal tissue and/or corneal limbus tissue, for example, in a human subject.

Another aspect of the invention is an isolated conjunctiva organoid, in particular a mammal, and more in particular a human conjunctiva organoid, comprising cells that express Keratin-19 (KRT19), and one or more of the transcription factor p63 (TP63), the mucin short variant S1 (MUC1), and the aquaporin-5 (AQP5).

Another aspect is an isolated limbal organoid, in particular a mammal, and more in particular a human limbal organoid, comprising cells that express Keratin-24 (KRT24), the transcription factor p63 (TP63), and the ocular surface transcription factor PAX6. Another aspect of the invention is an organoid obtained from culturing conjunctival stem-cells and limbal stem-cells at the same time, as indicated above.

The method according to which the conjunctiva stem-cells or the limbal stem-cells have been allowed to grow, may impart to the cells certain structural and functional features. Thus, the organoids can also be defined by their method of obtaining. For example, in the case of limbal stem cells, the organoids (or ALI cultures) obtained with the method of the invention may more resemble corneal tissue or may more resemble corneal limbus tissue depending on, for example, how long the culture is maintained and/or the differentiation state of the cells comprised. For example, it is contemplated that in ALI culture a culture more resembling cornea is obtained when the culturing is started based on a limbal organoid that already differentiated more in the direction of cornea; in case a less differentiated limbal organoid would be used in the ALI culture, the skilled person understand an ALI culture may be obtained wherein the tissue more resembles corneal limbus tissue. The skilled person understands, in view of the disclosure herein, how to achieve this.

Therefore, another aspect of the invention is an isolated mammal conjunctiva organoid obtainable or obtained by a method as defined in the first aspect. Therefore, another aspect of the invention is an isolated mammal limbal organoid obtainable or obtained by a method as defined in the first aspect.

Thus, the invention relates to an isolated mammal conjunctiva organoid or an isolated mammal limbal organoid obtained or obtainable by a method as defined in the previous aspect, in particular obtainable or obtained by
(a) providing conjunctival stem-cells or limbal stem-cells;
(b) culturing the conjunctival stem-cells or the limbal stem-cells in a culture medium, and under conditions suitable to form a conjunctiva organoid or to form a limbal organoid.

As indicated, herewith provided are also derivative products and devices comprising the organoids of conjunctiva epithelium of the invention and/or comprising the limbal organoids of the invention, which are useful tools for the analysis thereof, and for the test of compounds.

The invention provides, as another aspect, an air-liquid interface culture of conjunctiva cells, preferably conjunctiva organoid cells comprising:
I. a container comprising a first chamber and a second chamber, said first and second chambers separated by a porous membrane; and
II. a multi-layer of conjunctiva cells, in particular derived from dissociated conjunctiva organoids disposed on at least one side of the porous membrane, said multi-layer comprising at least one layer of cells proximal to the porous membrane and comprising basal keratinocytes that express KRT-19 and TP63; and at least one layer of cells distal to the support and comprising apical keratinocytes that express KRT-19, MUC1 and AQP5, and goblet cells, and wherein the air-liquid interface culture optionally comprises a cell culture medium in at least one of the first and second chamber.

The air-liquid interface culture of conjunctiva cells, preferably conjunctiva organoid cells, is a reliable and accurate model of the conjunctiva, in particular of the epithelium conjunctiva.

Indeed, as will be illustrated, pathological conditions of the conjunctiva were reproduced with the model, thus helping to study these conditions, and helping in the finding of therapeutic approaches.

The invention provides, as another aspect, an air-liquid interface culture of corneal epithelium and/or limbal epithelium, preferably limbal organoid cells, comprising:
III. a container comprising a first chamber and a second chamber, said first and second chambers separated by a porous membrane; and
IV. a multi-layer of cells comprising corneal epithelium and/or limbal epithelium, in particular derived from dissociated limbal organoids, disposed on at least one side of the porous membrane, said multi-layer comprising at least cells expressing Keratin-24 (KRT24), the transcription factor p63 (TP63), and the ocular surface transcription factor PAX6, and wherein the air-liquid interface culture optionally comprises a cell culture medium in at least one of the first and second chamber.

The air-liquid interface culture of corneal epithelium and/or limbal epithelium cells, preferably limbal organoid cells, is a reliable and accurate model of corneal limbus tissue and/or corneal tissue (including corneal epithelium and/or limbal epithelium).

Indeed, pathological conditions of corneal limbus tissue and/or corneal tissue (including corneal epithelium and/or limbal epithelium) can be reproduced with the model, thus helping to study these conditions, and helping in the finding of therapeutic approaches.

Yet another aspect of the invention is a conjunctiva implant, comprising, or consisting of, a conjunctiva organoid as defined in the previous aspects, or as obtained with a method as defined in the first aspect, or comprising or consisting of cells dissociated from said conjunctiva organoid. The conjunctiva implant is also herewith called cell implant, cell transplant or cell graft (transplant of living cells or tissue).

A direct application of this implant when properly transplanted in an animal or subject is its use as a medicament.

Thus, another aspect of the invention is an in vitro obtained conjunctiva organoid, or an in vitro obtained conjunctiva implant for use as a medicament, wherein the conjunctiva implant is as defined in the previous aspect, and/or wherein the in vitro obtained conjunctiva organoid is obtainable with a method as defined in the first aspect, and/or wherein the in vitro obtained conjunctiva organoid is as defined also in the previous aspects.

Yet another aspect of the invention is a corneal epithelium and/or limbal epithelium implant, comprising, or consisting of, a limbal organoid as defined in the previous aspects, or as obtained with a method as defined herein, or comprising or consisting of cells dissociated from said a limbal organoid. The corneal epithelium and/or limbal epithelium implant may also be referred to as a cell implant, cell transplant or cell graft (transplant of living cells or tissue).

A direct application of this implant when properly transplanted in an animal or subject is its use as a medicament.

Thus, another aspect of the invention is an in vitro obtained limbal organoid, or an in vitro obtained corneal epithelium and/or limbal epithelium implant for use as a medicament, wherein the implant is as defined in the previous aspect, and/or wherein the in vitro obtained limbal organoid is obtainable with a method as defined herein and/or wherein the in vitro obtained limbal organoid is as defined also in the previous aspects. The invention relates to the use of the organoids or of the air-liquid interface culture all as defined in the previous aspects, as an in vitro model of mammalian conjunctival epithelia or of mammalian corneal limbus tissue and/or corneal tissue (including corneal epithelium and/or limbal epithelium), or in drug discovery screening, or in toxicity assays.

With a reliable model of mammalian conjunctival epithelia, or of corneal limbus tissue and/or corneal tissue (including corneal epithelium and/or limbal epithelium), the screening of drug candidates and the analysis of parameters that can modulate the physiology of the conjunctiva corneal limbus tissue and/or corneal tissue (including corneal epithelium and/or limbal epithelium) are possible. Moreover, this model avoids the disadvantages associated to the use of short-term explants, or the disadvantages associated to the models derived from cancer cell lines, on, in a preferred embodiment, from induced pluripotent stem-cells.

Thus, it is also herewith provided, as a further aspect, a method for the screening of a candidate agent to modulate conjunctiva and/or to prevent and/or treat a disease or disorder of the conjunctiva, in particular human conjunctiva, comprising:
(a) providing a conjunctiva organoid as defined, or an air-liquid interface culture both as defined in the previous aspects;
(b) optionally, providing a condition or an agent to simulate the disease or disorder;
(c) providing a candidate agent;
(d) contacting the candidate agent with the conjunctiva organoid or the air-liquid interface culture under conditions that allow interaction of the candidate agent with the conjunctiva organoid or the air-liquid interface culture, optionally, prior, after, or simultaneously to step (b); and
(e) determining if the contacting with the candidate agent modulates conjunctiva (conjunctiva organoid or the air-liquid interface culture) and/or prevents or reverts the simulated disorder or disease.

For the carrying out of the method of the invention for producing conjunctiva organoids, in particular mammal conjunctiva organoids, novel cell culture mediums were developed by the inventors.

Thus, it is also herewith provided, as a further aspect, a method for the screening of a candidate agent to modulate corneal epithelium and/or limbal epithelium and/or to prevent and/or treat a disease or disorder of corneal limbus tissue and/or corneal tissue (including corneal epithelium and/or limbal epithelium), in particular human tissue comprising:
(a) providing a limbal organoid as defined, or an air-liquid interface culture both as defined in the previous aspects;
(b) optionally, providing a condition or an agent to simulate the disease or disorder;
(c) providing a candidate agent;
(d) contacting the candidate agent with the limbal organoid or the air-liquid interface culture under conditions that allow interaction of the candidate agent with the limbal organoid or the air-liquid interface culture, optionally, prior, after, or simultaneously to step (b); and
(e) determining if the contacting with the candidate agent modulates the limbal organoid or the air-liquid interface culture and/or prevents or reverts the simulated disorder or disease.

For the carrying out of the method of the invention for producing conjunctiva organoids or limbal organoids, in particular mammal conjunctiva organoids or mammalian limbal organoids, novel cell culture mediums were developed by the inventors.

Thus, another aspect of the invention is a cell culture medium, preferably a cell expansion culture medium, comprising a basal medium for mammal cells supplemented with an inhibitor of the transforming growth factor beta (TGF-β) signaling pathway, an inhibitor of the bone morphogenic protein (BMP), and an agonist of the Wnt pathway, and optionally supplemented with one or more of B27 supplement; N-acetylcysteine (NAC); a fibroblast growth factor (FGF), in particular selected from FGF10, FGF1, FGF2 and FGF7 and combinations thereof; an epidermal growth factor (EGF); and an antibacterial and/or antifungal compound.

In some preferred embodiments, the culture medium further comprises forskolin or FGF-10 or WNT surrogate, or forskolin and FGF-10, or, preferably, forskolin, FGF-10 and WNT surrogate.

In some more preferred embodiments, the culture medium further comprises (e.g. next to forskolin or FGF-10 or WNT surrogate, or forskolin and FGF-10, or, preferably, forskolin, FGF-10 and WNT surrogate), IGF1 or FGF7, or, in a preferred embodiment IGF1 and FGF7. In yet further embodiments, the culture medium further comprises IL-6, for example, in combination with IGF1 and FGF7, for example in combination with forskolin, FGF-10 and WNT surrogate (or Wnt agonist).

Yet another aspect is a cell culture medium, preferably a cell differentiation culture medium comprising basal medium for mammal cells supplemented with an inhibitor of the transforming growth factor beta (TGF-β) signaling pathway, an inhibitor of the bone morphogenic protein (BMP), and an agonist of the Wnt pathway, and which medium is substantially free of epidermal growth factor (EGF), is substantially free of any fibroblast growth factor (FGF) and is substantially free of B27 supplement.

### BRIEF DESCRIPTION OF THE DRAWINGS

Embodiments of the invention are further described hereinafter with reference to the accompanying drawings, in which:

FIG. 1: (A) Schematic of mouse conjunctival organoid establishment. (B) Brightfield images of organoid outgrowth and organoid morphology after 34 passages. Scale bars, 500 μm (4× pictures) and 100 μm (20× pictures). (C) Immunohistochemistry analysis for the indicated markers of mouse conjunctiva organoids compared to mouse conjunctival tissue. Scale bars, 50 μm. Insets scale bars, 20 μm. (D) Schematic of the establishment of Pax6 knock-out (Pax6KO). (E) Sequencing traces of 3 Pax6KO clones. A fragment of Pax6 Mus musculus, exon 4 is depicted,

(SEQ ID NO: 17)
TGCCGGACTCCACCCGGCAGATCGTAGAGCTA.

(F) Brightfield images of representative WT and Pax6KO clones at respectively p32 and p25 13 days after split. Scale bars, 500 μm. (G) Volcano plot of the differentially expressed genes between WT (n=2 lines) and Pax6KO (n=3 lines) organoids. In light grey are indicated the genes with fc>2 and p-adj<0.01. (H) Staining for PAX6, TP63 and PAS in WT and Pax6KO clone 5. Scale bars, 50 μm; insets, 20 μm.

FIG. 2: Establishment and characterization of human conjunctiva organoids and air-liquid interface cultures. (A) Schematic of human conjunctival organoid establishment. (B) Brightfield images of organoid outgrowth and organoid morphology. Scale bars, 500 μm (4× pictures) and 100 μm (20× pictures). (C) Immunohistochemistry analysis for the indicated markers of human conjunctiva organoids compared to tissue. Scale bars, 50 μm. (D) Schematic of human conjunctival organoid differentiation. (E) Immunohistochemistry analysis for the indicated markers of human conjunctiva organoids cultured in expansion and differentiation media. Scale bars, large 200 μm, inset 50 μm.

FIG. 3: Organoid-derived air-liquid interface culture promotes Goblet cell differentiation. (A) Schematic of human conjunctival air-liquid interface cultures. (B) Brightfield images of air-liquid interface over the course of 17 days after lifting to ALI. Scale bars, 50 μm. (C) Immunohistochemistry of the indicated markers of human conjunctiva ALI cultures 4 and 17 days after shifting to ALI. Scale bars, 50 μm. (D) Transmission electron microscopy of a Goblet cell in a 17-day-old ALI culture (left) and in primary tissue (right). Scale bars, 5 μm.

FIG. 4: Characterization of epithelial cell types of the conjunctiva compared to culture models. (A) Schematic of the different samples subjected to single-cell RNA sequencing. (B) and (C). Immunohistochemistry staining for LCN2 (B) and WFDC2 (C) in conjunctival tissue and 17-day-old ALI cultures. Scale bars, 50 μm. (D) Electron microscopy image of 17-day-old ALI culture. Arrowheads point at vesicles present at the apical surface of the ALI culture. Left scale bar, 1 μm; right, 200 nm. (E) LCN2 secretion assay was performed on ALI cultures 1, 4, 10, 17 and 22 days after lifting to ALI. The supernatant secreted for 24 hours was collected, loaded on a protein gel (Western blot), and stained for LCN2.

FIG. 5: NGFR+ cells are bipotent conjunctival stem-cells. (A) NGFR expression in the single-cell dataset. (B) Brightfield images (left) and quantification (right) of organoid outgrowth from NGFR− and NGFR+ cells. Scale bars, 500 μm. Each dot represents an independent experiment. (C) Staining for the Goblet cell marker MUC5AC and the keratinocyte marker MUC1 in a single-cell-derived organoid. Arrowheads point at cells that are positive for either marker. Scale bars, 100 μm.

FIG. 6: Conjunctival air-liquid interfaces sustain HSV1, hAdV8 and SARS-CoV-2 infection. (A) Schematic of HSV1 infection protocol. One of HSV1 capsid protein is tagged with tdTomato. (B) HSV1 titers in conjunctival ALI cultures detected by qPCR, with and without 10 μM Acyclovir treatment. Supernatants were collected every 24 hours to assess the number of HSV1 genome copies. n=3 independent experiments. (C) Schematic of SARS-COV-2 infection protocol. (D) SARS-COV-2 variants 614G and Delta titers in conjunctival ALI cultures detected by qPCR. Supernatants were collected every 24 hours to assess the number of SARS-COV-2 RNA copies. n=3 independent infected ALI cultures. (E) Schematic of hAdV8 infection protocol. (F) Brightfield images of ALI cultures 48 hours (top) and 96 hours (bottom) following hAdV8 infection with or without 10 μM Acyclovir, 60 μM Cidofovir or 20 μM Nelfinavir highlighting the appearance of cytopathic effects. Scale bars, 100 μm. (G) hAdV8 titers in conjunctival ALI cultures detected by qPCR, with and without 10 μM Acyclovir, 60 μM Cidofovir or 20 μM Nelfinavir treatment. Supernatants were collected every 24 hours to assess the number of hAdV8 genome copies. n=3 independent experiments.

FIG. 7: Human conjunctival organoids can engraft and be engineered for human transplantation. (A) Schematic of organoid transplantation in NSG mice. (B) Staining for human KRT19 (hKRT19), KI67, TP63 and MUC1 in a transplanted eye 2 days after the surgery. Scale bars; large panel 500 μm, small panels 50 μm. Representative of n=2 mice. (C) Staining for hKRT19, human nucleoli, MUC5AC, MUC1 and TP63 3 weeks after transplantation. Dashed lines underline the graft location based on human stainings on immediately consecutive sections. Scale bars, left panels 100 μm, right panels 50 μm. (D) Quantification of successful engraftment after 3 weeks.

FIG. 8: Engineering of a transplantation-ready conjunctival cell sheet on fibrin substrate. (A) Schematic of human organoid-based cell sheet engineering ready for transplantation. (B) Staining for human KRT19 (hKRT19), TP63 and MUC1 7 days after seeding. Scale bars; large panel 1 mm, small panels 50 μm.

FIG. 9: Organoid outgrowth on a collagen I matrix. Scale bars, overviews: 500 μm, zoom-ins: 100 μm, except for the top right inset, 500 μm.

FIG. 10: This photograph shows the region of the defect with a dashed white line (5 mm diameter). The sutures that marked the boundaries of the graft are clearly visible, which is an indication of how well the defect healed.

FIG. 11: The results show that the defect possesses cells of human (i.e., organoid) origin with many Goblet cells present.

FIG. 12: A: Limbal organoids obtained with the method according to the invention. B: Human limbal organoid passing number in expansion medium (hCJ), X2 medium and X3 medium for the limbal organoid line hCo8. Culturing and passaging is still continued. Comparable results were obtained with other limbal organoid lines.

FIG. 13: 2D ALI cultures of limbal organoids.

### DESCRIPTION

### Definitions

A portion of this disclosure contains material that is subject to copyright protection (such as, but not limited to, diagrams, device photographs, or any other aspects of this submission for which copyright protection is or may be available in any jurisdiction.). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

For purposes of the present invention, the following terms are defined below.

As used herein, the singular forms “a,” “an” and “the” include plural referents unless the context clearly dictates otherwise. The indefinite articles “a” and “an” are synonymous with “at least one” or “one or more”.

As used herein, the term “amount” is used interchangeably with the term “dose”.

As used herein, the term “and/or” indicates that one or more of the stated cases may occur, alone or in combination with at least one of the stated cases, up to with all of the stated cases.

As used herein, the term “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

As used herein, “comprising” or “to comprise” is construed as being inclusive and open ended, and not exclusive. Specifically, the term and variations thereof mean the specified features, steps or components are included. These terms are not to be interpreted to exclude the presence of other features, steps, or components. It also encompasses the more limiting “to consist of”.

As used herein, “conventional techniques” or “methods known to the skilled person” refer to a situation wherein the methods of carrying out the conventional techniques used in methods as disclosed herein will be evident to the skilled worker. The practice of conventional techniques in molecular biology, biochemistry, cell culture, genomics, sequencing, medical treatment, pharmacology, immunology, and related fields are well-known to those of skill in the art and are discussed, in various handbooks and literature references.

As used herein, the term “subject” refers to any vertebrate animal, but will typically pertain to a mammal, for example a human, a domesticated animal (such as dog or cat), a farm animal (such as horse, cow, or sheep) or a laboratory animal (such as rat, mouse, non-human primate or guinea pig, rabbit). In preferred examples, the subject is human and includes males, females, adult, elderly, children, or infants, in the need of treatment, in particular in the need of treatment involving transplantation of (part of) the conjunctiva.

As used herein, the term “therapeutically effective amount” or “effective amount” refers to the amount of an organoid, a graft containing the organoid, the cells making up the organoid or the graft containing the organoid, or any other product as disclosed herein, which is effective for producing an effective and desired (therapeutic) effect in a subject at a reasonable benefit/risk ratio applicable and within the context of the treatment of the invention.

As used herein, the terms “treatment” and “treating” refer to therapeutic treatment. The object of the treatment is to at least slow down the disease condition. Those in need of the treatment include those already with the disease condition.

An “organoid” is an in vitro obtained structured cell system that reproduces and mimics a miniaturized and simplified version of an actual organ, in terms of the key functional, structural, and biological complexity of that organ. Organoids may be obtained by culturing stem-cells embedded in a 3D cell culture medium, which generally is a hydrogel that contains extracellular matrix components. Thus, “conjunctival organoids” are organoids that mimic the actual, in particular mammal, conjunctiva. The conjunctival organoids of this invention are, more specifically, conjunctival epithelium organoids, which means that they mimic the conjunctival epithelium. The organoids are obtained from conjunctiva stem-cells, which are the basal cells found in the lower layers of multi-layered epithelium and pockets in single-layered epithelium. Thus, “limbal organoids” are organoids that mimic the actual, in particular mammal, corneal limbus tissue and/or corneal tissue (including corneal epithelium and/or limbal epithelium). The organoids are obtained from limbal stem-cells, which are the cells found in the corneal limbus and/or corneal tissue. It will thus be understood by the skilled person that in the context of the current invention, terms such as corneal limbus tissue, corneal tissue (including corneal epithelium and/or limbal epithelium) are related in that they are obtainable with the method disclosed herein, based on the use of the limbal stem-cells and/or limbal organoids.

“Conjunctival stem-cells”, also herewith referred as adult conjunctival stem-cells, or “basal cells” are to be understood as those at certain extent undifferentiated cells that, upon proper stimulation or conditions, can differentiate to the more specialized cells found in the conjunctiva epithelium, such as keratinocytes and Goblet cells. In this description “basal cells” are also identified as the cells in the organoids that express the transcription factor TP63. “Basal cell” in the epithelium structure is commonly recognized as an epithelial stem-cell but can also refer to any cell that sits on an epithelial basement membrane (i.e., basally located). In this description basal cells are the cells outside the surface of organoids that contact the base membrane extract or fibrin in the cell cultures herewith disclosed. They mimic the cells located in the lower layers (i.e., basally located) of the multi-layered epithelium and pockets in single-layered epithelium and constitute the so-called stratum basale. “Limbal stem-cells”, also referred to as adult limbal stem-cells or corneal epithelial stem-cells, are to be understood as those at certain extent undifferentiated cells that, upon proper stimulation or conditions, can differentiate to the more specialized cells found in corneal epithelium and/or limbal epithelium, for example as found in the cornea and/or corneal limbus.

“Keratinocytes” are the main differentiated cells found in the conjunctival epithelium. They differentiate from stem-cells (i.e., the basal stem-cells) in the lower part of the epithelium and migrate towards the surface. Different markers are expressed in keratinocytes depending on the stage of differentiation, but there are mainly keratins and mucins. In the organoids herewith disclosed, keratinocytes are referred as the differentiated conjunctival epidermal cells that preferably do not express TP63 and/or that express at least mucin 1 (MUC1) and aquaporin 5 (AQP5). They are sited at the apical and middle zones of the organoids, and in the apical or middle layers of a cell culture on a plate. In the organoids derived from the conjunctiva stem-cells allowed to expand and/or proliferate and/or differentiate in a hydrogel matrix that mimics the extracellular cell matrix (i.e., the base membrane extract or fibrin), the apical zone is the area located in the organoid not in contact with this matrix (i.e., not in contact with the fibrin or basement membrane extract). In a multi-layered cell culture on a plate (i.e., an ALI culture) obtained from the expansion and/or proliferation and/or differentiation of the conjunctiva stem-cells, the apical zone is the area defined by the layers of cells that are located not in contact with the support or matrix (i.e., not in contact with the base membrane extract or fibrin). Therefore, the apical or middle zones of the organoids, or of a multi-layered in vitro cell culture of conjunctiva, correspond to the areas far from or not in contact with the support or with the matrix that mimics the extracellular cell matrix, and upon which or wherein the conjunctiva stem-cells were initially seeded and were allowed to expand and/or proliferate and/or differentiate.

“Goblet cells” are also specialized (i.e., differentiated) cells scattered in among the conjunctival epithelia. Morphologically they are columnar and secrete mucins. In this description they are identified as expressing, in particular mucin 5AC (MUC5AC) and disposed apically in relation to the support or matrix in which the organoids are formed.

When in the present invention the expression “isolating from conjunctiva” or “isolating from cornea” or “isolating from corneal limbus” is used, it refers to encompass the minimal manipulation of a biopsy, such as simply contacting a biopsy with a mimic of the extracellular matrix (e.g., BME) and adding the (expansion) cell culture medium, or the homogenization of the biopsy to obtain a mixture of single-cells or cell aggregates. It also encompasses a more complex manipulation, in which the different cells in the conjunctiva, cornea or corneal limbus, are selectively separated, and a fraction comprising or consisting in conjunctival stem-cells (present in the conjunctiva tissue) or limbal stem-cells (present in the corneal limbus tissue and/or corneal tissue), respectively, is isolated to be cultured as indicated.

The expression “substantially free”, when in the present description is referred to the presence and amount of a compound or composition, it is to be understood as the compound or composition no being comprised in the medium, but also as including those amounts (minor amounts) that do not disturb the growth and lifespan of the organoids, or that do not disturb the effect aimed with a particular medium or environment defined as substantially free of the compound or composition.

As used herein the “expansion medium” refers to a cell culture medium that promotes the growth of the organoids in terms of extending their surface area, while they are also proliferating in number of cells in a basal cell culture medium with the nutrients for the maintenance of cell life.

The expansion medium used in the method disclosed herein surprisingly allows for the long-term culturing of, respectively, the conjunctiva organoid and/or limbal organoid, without losing the ability to be split (or passaged). In other words, the expansion medium according to the invention surprisingly maintains the stem-cell potency of the culture, without the cells losing the ability to expand over prolonged periods of culturing (or passaging of the cells). For example, in some embodiments, the organoids according to the invention may be passaged (or split) for at least 10 times or more, without the cell culture losing the ability to continue expanding. This, for example, allows the continue culturing of the organoids, starting from, for example, stem-cells obtained from a biopsy, for a prolonged period of time, for example, for at least 50 days, or 100 days or more.

The “differentiation medium”, as referred in this description, is to be understood as a medium that promotes stem-cells to change from a less specialized type or stage to a more specialized in form and function. In the particular case of the invention, it is a medium that comprises one or more compounds that allow the cells to propagate and then to capacitate (i.e., to start differentiation towards keratinocytes, goblet cells and tuft cells in the case of conjunctiva).

The skilled person in the art know these differentiation culture media from the prior art. In this description, particular useful examples to promote differentiation of conjunctival organoids, that may likewise be useful for differentiation of limbal organoids, are provided. As used herein “candidate agent” or “agent” refers to a molecule that may be screened for, or be identified as, modulating the development of, respectively, the conjunctiva epithelium, or corneal epithelium and/or limbal epithelium, its regeneration after damage or its permeability to the tested agent or to other substances. Such agent may, for example, be an inhibitor or enhancer (i.e., promoter) of, respectively, the conjunctiva functionality or corneal limbus tissue and/or corneal tissue (including corneal epithelium and/or limbal epithelium) functionality, and may find use in a variety of applications, including therapy.

The screening methods will typically be assays which provide for qualitative/quantitative measurements of the activity (i.e., modulation of conjunctiva development, permeability, regeneration, or modulation of corneal limbus tissue and/or corneal tissue (including corneal epithelium and/or limbal epithelium) development, permeability, regeneration, or modulation in the presence of a particular candidate agent. In embodiments of the invention, the screening is using conjunctiva organoids. In embodiments of the invention, the screening is using limbal organoids. In embodiments of the invention, the screening is using ALI cultured conjunctiva as disclosed herein (e.g. obtained from conjunctiva organoids). In embodiments of the invention, the screening is using ALI cultured corneal limbus tissue and/or corneal tissue (including corneal epithelium and/or limbal epithelium) as disclosed herein (e.g. obtained from limbal organoids herein).

(Candidate) agents may be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts or purified compounds are available or may be produced. Additionally, natural, or synthetically produced libraries and compounds can be prepared using conventional chemical, physical and biochemical means, and may be used to produce combinatorial libraries. Known pharmacological agents may be subjected to directed or random chemical modifications, such as acylation, alkylation, esterification, amidification, etc. to produce structural analogues or derivates. (Candidate) agents may also be biomolecules including peptides, saccharides, fatty acids, steroids, purines, pyrimidines, derivatives, structural analogues, or combinations thereof.

### DETAILED DESCRIPTION

The invention is defined herein, and in particular in the accompanying claims. Subject-matter which is not encompassed by the scope of the claims does not form part of the present claimed invention.

It is contemplated that any method, use, product, or composition described herein can be implemented with respect to any other method, use, product, or composition described herein. Embodiments discussed in the context of methods, use, products and/or compositions of the invention may be employed with respect to any other method, use, product, or composition described herein. Thus, an embodiment pertaining to one method, use, product, or composition may be applied to other methods, uses, products and compositions of the invention as well.

Any references in the description to methods of treatment refer to the compounds, pharmaceutical compositions, and medicaments of the present invention for use in a method for treatment of the human (or animal) body by therapy.

As embodied and broadly described herein, the present invention is directed to the surprising finding that long-term growth organoids of the conjunctiva epithelium, or of the primary conjunctival tissue, can be obtained under reliable and reproducible conditions from conjunctiva stem-cells. The organoids recapitulate the essential structural and physiological features of actual conjunctiva epithelium. They are obtained in sufficient amounts or with the appropriate surface area to be used in the replacement or refilling of actual conjunctiva in a mammal, in particular in a human.

Likewise, as embodied, and broadly described herein, the present invention is directed to the surprising finding that long-term growth of limbal organoids, or of corneal epithelium and/or limbal epithelium, or of the primary corneal limbus tissue and/or corneal tissue, can be obtained under reliable and reproducible conditions from limbal stem-cells. The organoids recapitulate the essential structural and physiological features of actual corneal limbus tissue and/or corneal tissue (including corneal epithelium and/or limbal epithelium). They are obtained in sufficient amounts or with the appropriate surface area to be used in the replacement or refilling of corneal limbus tissue and/or corneal tissue (including corneal epithelium and/or limbal epithelium), in particular in a human.

Considering the aimed practical uses of the organoids, namely as study models of the physiology and architecture of organs and tissues, as well as their applicability as tissue or organ replacers, it is of relevance that they have a long lifespan for their manipulation, and also for the engraftment in a receptor body.

The finding is unexpected, since by now most of the attempts to obtain conjunctival epithelium related to short-lived explants, which carried the inherent problems of the possibility to obtain enough tissue and maintain it stable and viable until their use in case of a transplant. When conjunctiva organoids were obtained from the expansion of iPSC, the obtained conjunctival epithelium did not recapitulate the maturity of an actual conjunctival epithelium, which makes them non adequate enough as conjunctiva models, or that questions their use as transplants. Likewise, attempt to obtain limbal organoids have only been partially successful, suffering from the same type of problems indicated above.

Therefore, the invention herewith disclosed also supposes the provision of a solution for an unmet need in the field, which was lacking a versatile platform to study conjunctival (patho-)physiology, and of in vitro produced conjunctival tissue that recapitulates all key features of the conjunctival epithelium.

Likewise, therefore, the invention herewith disclosed also supposes the provision of a solution for an unmet need in the field, which was lacking a versatile platform to study limbal and corneal (patho-)physiology, and of in vitro produced limbal, e.g. corneal limbus tissue and/or corneal tissue, preferably corneal epithelium and/or limbal epithelium tissue, that recapitulates all key features of corneal epithelium.

### In Vitro Method for the Production of Conjunctiva Organoids or Limbal Organoids:

As previously indicated, the invention relates first to an in vitro method for producing a conjunctiva organoid, in particular a mammal conjunctiva organoid, comprising the steps of:
(a) providing conjunctival stem-cells, in particular from an isolated conjunctiva sample;
(b) culturing the conjunctival stem-cells in a culture medium, and under conditions suitable to form a conjunctiva organoid.

As previously indicated, the invention relates first to an in vitro method for producing a limbal organoid, in particular a mammal limbal organoid, comprising the steps of:
(a) providing limbal stem-cells, in particular from an isolated corneal limbus or cornea sample;
(b) culturing the limbal stem-cells in a culture medium, and under conditions suitable to form a limbal organoid.

In a particular embodiment of the in vitro method as disclosed above, the organoid is a conjunctival epithelium organoid. In a particular embodiment of the in vitro method as disclosed above, the organoid is a limbal (or corneal epithelium) organoid.

In a particular embodiment of the in vitro method as disclosed above, culturing the conjunctival stem-cells or the limbal stem-cells in a culture medium comprises culturing in a cell expansion culture medium, preferably wherein the cell expansion culture medium comprises an inhibitor of the transforming growth factor beta (TGF-β) signaling pathway, an inhibitor of the bone morphogenic protein (BMP), and/or an agonist of the Wnt pathway.

In a particular embodiment of the in vitro method as disclosed above, culturing the conjunctival stem-cells or the limbal stem-cells in a culture medium comprises culturing in a cell expansion culture medium, preferably wherein the cell expansion culture medium comprises an inhibitor of the transforming growth factor beta (TGF-β) signaling pathway, an inhibitor of the bone morphogenic protein (BMP), and/or an agonist of the Wnt pathway, a cyclic AMP activator, and/or an activator of fibroblast growth factor (FGF) signaling.

In a particular embodiment of the in vitro method as disclosed above, culturing the conjunctival stem-cells or the limbal stem-cells in a culture medium comprises culturing in a cell expansion culture medium, preferably wherein the cell expansion culture medium comprises an inhibitor of the transforming growth factor beta (TGF-β) signaling pathway, an inhibitor of the bone morphogenic protein (BMP), an agonist of the Wnt pathway, a cyclic AMP activator, and an activator of fibroblast growth factor (FGF) signaling, in particular one or more fibroblast growth factor(s) (FGF), in particular FGF-1 and/or FGF-10, preferably FGF1 and FGF10.

In another particular embodiment of the cell culture medium, preferably cell expansion culture medium, it further comprises interleukins, in particular one or more of interleukin-4 (IL4) and interleukin-13 (IL-13), more preferably interleukin-6 (IL-6).

A cell culture medium, preferably cell expansion culture medium, comprising the interleukins allow to mimic inflammatory conditions, which can be used to study the conjunctiva under this challenge.

Moreover, the presence of the interleukins, preferably one or more of IL-4 and IL-13, in the cell culture medium, preferably cell expansion culture medium, do also enhances the expansion of the goblet cells ad tuft cells in the conjunctiva organoid.

In another particular embodiment of the cell culture medium, preferably cell expansion culture medium, it further comprises an insulin-like growth factor, in particular insulin-like growth factor 1 (IGF-1) (or derivates thereof such as the Long R3 recombinant).

In another particular embodiment of the cell culture medium, preferably cell expansion culture medium, it further comprises keratinocyte growth factor, also referred to as FGF-7.

In a preferred embodiment the culture medium, in particular the expansion medium comprises IGF-1 and FGF-7. Therefore, in a particular preferred embodiment the culture medium, in particular expansion medium comprises an inhibitor of the transforming growth factor beta (TGF-β) signaling pathway, an inhibitor of the bone morphogenic protein (BMP), an agonist of the Wnt pathway, a cyclic AMP activator, an activator of fibroblast growth factor (FGF) signaling (in particular one or more fibroblast growth factor(s) (FGF), in particular FGF-1 and/or FGF-10), preferably FGF1 and FGF10, IGF-1 and FGF-7.

In another particular embodiment, the in vitro method comprises culturing in the culture medium for a period of time sufficient to allow formation of the conjunctiva organoids, preferably wherein the formed conjunctiva organoids comprise cells that express one or more of the transcription factor Tumor protein p63 (TP63), Keratin-19 (KRT19), mucin short variant S1 (MUC1), and aquaporin-5 (AQP5).

More in particular, the formed conjunctiva organoids comprise cells that express KRT19, and one or more of TP63, MUC1, and AQP5.

Even more in particular, the formed conjunctiva organoids comprise basal cells that express KRT19, and TP63; and differentiated apical keratinocytes that express KRT19, and one or more of MUC1 and AQP5, in particular both of MUC1 and AQP5.

As indicated, the spatial distribution of these cells, and so their attributes as basal or apical, mimic the ones of the actual epithelium conjunctiva. The basal location corresponds to the outside surface of the organoid in contact with a fibrin or basement membrane extract matrix, or collagen, which mimic the extracellular matrix, and that are used to culture the cells therein or thereupon. The basal cells are indeed conjunctiva stem-cells. The apical location, mainly occupied by the keratinocytes, corresponds to the area in the organoid not in contact with the fibrin or basement membrane extract.

In another particular embodiment, the in vitro method comprises culturing in the culture medium for a period of time sufficient to allow formation of the limbal organoids, preferably wherein the formed limbal organoids comprise cells that express one or more of the transcription factor Tumor protein p63 (TP63), Keratin-24 (KRT24), and PAX6 (Paired box protein Pax-6, also known as aniridia type II protein (AN2) or oculorhombin). In a preferred embodiment, the formed limbal organoids comprise cells that express KRT12. In some embodiments the cells do not or not substantially express MUC-1 (Mucin-1).

In another particular embodiment of the in vitro method for the production of conjunctiva organoids and/or limbal organoids, the culturing in culture medium is for a period of at least 3 days. More in particular it is for a period selected from 3 days to 10 weeks, 3 days to 8 weeks, 3 days to 7 weeks, 3 days to 6 weeks, 3 days to 5 weeks, 3 days to 4 weeks, 3 days to 3 weeks, 3 days to 2 weeks, and 3 days to 1 week. In another more particular embodiment, it is for a period selected from 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, and 56 days. The skilled person understands that during the culturing, for example in the expansion medium, culture medium may be replaced by fresh culture medium. It was surprisingly found that with the expansion medium disclosed herein it has now become possible to culture conjunctiva organoids and/or limbal organoid for an extended period of time, for example for at least 60, 70, 80, 90, 100 days, 200 days or more.

It was therefore also surprisingly found that with the culture method according to the invention, using the expansion medium of the invention, the organoids can be passaged (split) for a prolonged period of time, for example, at least 10, 20, 30, 40, 50 or more passages can be achieved for the conjunctiva organoids and, for example, at least 10, 20, 30, 40, 50 or more passages can be achieved for the limbal organoids.

In another particular embodiment of the in vitro method for the production of conjunctiva organoids or limbal organoids, the culturing in culture medium is carried out on or in a composition comprising extracellular matrix compounds or a composition that mimics the extracellular matrix. These are well-known to the skilled person. In particular a hydrogel suitable for culturing the cells according to the invention is used.

Examples of compositions that resemble the extracellular matrix are hydrogels that comprise, among others a fibrin, a basement membrane extract matrix, collagen, or laminin. The skilled person will know the commercial compounds or compositions that mimic the extracellular matrix, and that are used in the conventional techniques for the culturing of cells, and that included, for example basal membrane extracts (BME) and Matrigel. In one embodiment, as shown in the examples, the conjunctival stem-cells and/or the limbal stem-cells are mixed with a non-solidified form of the hydrogel (for example basal membrane extract), after which the hydrogel is allowed to form a gel, allowing the cells to growth within the solidified hydrogel. Thus, in some embodiments the conjunctival stem-cells and/or the limbal stem-cells are present in the hydrogel, for example BME. In other examples, the conjunctival stem-cells and/or the limbal stem-cells are placed on top of the hydrogel, for example BME, or both.

In particular preferred embodiments, the conjunctival organoids or ALI cultures and/or the limbal organoids and/or ALI cultures can be grown on top of a collagen matrix, for example, at least for 1, 2 or more passages, for example 2 passages. This surprising effect allows for circumstances wherein it may not be desired to grow organoids in animal-derived matrices, for example when the conjunctival organoids or ALI cultures and/or the limbal organoids and/or ALI cultures would be used in transplantation.

In another particular embodiment of the in vitro method for the production of conjunctiva organoids, the conjunctiva is a mammalian, rodent or human conjunctiva, preferably a human conjunctiva. In another particular embodiment of the in vitro method for the production of limbal organoids, the cells are mammalian, rodent or human cells, preferably human cells.

In another particular embodiment of the in vitro method for the production of conjunctiva organoids, the conjunctival stem-cells are indeed the known as adult conjunctival stem-cells, in particular mammal adult conjunctival stem-cells. More in particular, they are human adult conjunctival stem-cells. In another particular embodiment of the in vitro method for the production of limbal organoids, the limbal stem-cells are indeed the known as adult limbal stem-cells, in particular mammal adult limbal stem-cells. More in particular, they are human adult limbal stem-cells.

For the provision of the conjunctival stem-cells and/or the limbal stem-cells, in particular adult conjunctival stem-cells and/or adult limbal stem-cells, different sources the skilled person will be aware of, or will know. A particular preferred source is from a primary tissue or sample thereof (biopsy) comprising these cells.

A particularly preferred source for the conjunctival stem-cells is the conjunctiva, in particular the mammal conjunctiva, and more in particular human conjunctiva, in which conjunctival adult stem-cells can be found in the same way as occurs for the other tissues of the body. The isolation or provision of the adult stem-cells from a conjunctiva sampling (i.e., biopsy) is of particular interest when the organoid is to be used as an autologous cell therapy. These organoids derived from the own conjunctiva will rarely activate the subject immune system against, for example, a graft constituted or comprising the organoids.

In also another more particular embodiment of the in vitro method of the invention, the conjunctiva is selected from one or more of palpebral conjunctiva and bulbar conjunctiva.

A particularly preferred source for the limbal stem-cells is corneal limbus tissue and/or corneal tissue, in particular mammal corneal limbus tissue and/or corneal tissue, and more in particular human corneal limbus tissue and/or corneal tissue, in which limbal adult stem-cells can be found in the same way as occurs for the other tissues of the body. The isolation or provision of the adult stem-cells from a corneal limbus or a cornea sampling (i.e., biopsy) is of particular interest when the organoid is to be used as an autologous cell therapy. These organoids derived from the own tissue will rarely activate the subject immune system against, for example, a graft constituted or comprising the organoids.

Advantageously, and as will be illustrated in the examples below, organoids that recapitulate the architecture and physiology of actual conjunctival epithelium tissue were obtained from either palpebral or bulbar conjunctiva. Advantageously, and as illustrated in test performed by the inventors, organoids that recapitulate the architecture and physiology of actual corneal limbus tissue and/or corneal tissue (including corneal epithelium and/or limbal epithelium) can be obtained from limbal stem-cells obtained from corneal limbus tissue and/or corneal tissue, comprising limbal adult stem-cells.

Another embodiment of the in vitro method according to the invention relates to a method wherein the conjunctival or limbal stem-cells, in particular adult stem-cells, are human conjunctival or limbal stem-cells, and, preferably, wherein the culture medium/cell expansion culture medium is substantially free of epidermal growth factor (EGF) and, optionally, comprises one or more of a cyclic AMP activator, in particular forskolin, and/or one or more fibroblast growth factor(s) (FGF), in particular selected from FGF1 and FGF10, and combinations thereof.

The inventors observed that when EGF is present in the cell culture medium, the lifespan of human conjunctival epithelium organoids or of the human limbal organoids, was reduced. Thus, it is not detrimental since it still allows for organoids outgrowth and expansion for several passages but makes less practical the method of production of the organoids. Thus, when in the present description the expression “substantially free” is referred to the presence and amount of EGF, it is to be understood as the compound no being comprised in the medium, but also as including those amounts (minor amounts) that do not disturb the growth and lifespan of the organoids.

The inventors have surprisingly found that a specific stem-cells of the conjunctiva, in particular of the mammal conjunctiva, were able to expand and differentiate under conditions allowing for the production of organoids comprising basal cells that besides KRT19 preferably express TP63; apical keratinocytes, that also besides KRT19 also express one or more of MUC1 and AQP5, and goblet cells, particularly expressing MUC5AC. These bipotent stem-cells, in terms that they can differentiate to either keratinocytes as well as to goblet cells, are the conjunctival stem-cells that express at least the nerve growth factor receptor (NGFR). Thus, they are NGFR positive cells.

Therefore, in another particular embodiment of the in vitro method according to the first aspect of the invention, the conjunctival stem-cells, preferably the human conjunctival stem-cells, express NGFR or are nerve growth factor receptor positive conjunctival stem-cells (NGFR+). It is contemplated the limbal stem-cells may express NGFR as well.

In another particular embodiment, the in vitro method for producing conjunctiva organoids or limbal organoids, comprises culturing the conjunctival stem-cells or organoids, or the limbal stem-cells or organoids, in a culture medium which is a cell differentiation culture medium, and preferably wherein the cell differentiation culture medium comprises an inhibitor of the TGF-β signaling pathway, an inhibitor of the BMP, and/or an agonist of the Wnt pathway, and even more preferably, wherein the cell differentiation culture medium is further substantially free of EGF, fibroblast growth factors, and/or B27 supplement.

In a preferred embodiment of the cell culture differentiation medium, the medium further comprises an insulin-like growth factor, in particular insulin-like growth factor 1 (IGF-1) (or derivates thereof such as the Long R3 recombinant). In another particular embodiment of the cell culture differentiation medium, it further comprises keratinocyte growth factor, also referred to as FGF-7. In a preferred embodiment the cell culture differentiation medium comprises IGF-1 and FGF-7. In some embodiments, the cell culture differentiation medium, in addition to IGF-1, FGF-7, or both, comprises IL-6.

Therefore, in a particular preferred embodiment the cell culture differentiation medium comprises an inhibitor of the transforming growth factor beta (TGF-β) signaling pathway, an inhibitor of the bone morphogenic protein (BMP), an agonist of the Wnt pathway, a cyclic AMP activator, IGF-1, and FGF-7. Preferably, the cell culture differentiation medium does not comprise FGF-1, FGF-10, or both. Preferably, in some embodiments, the cell culture differentiation medium does not comprise the B27 supplement, however its other embodiments the B27 supplement may be included. Preferably, the cell culture differentiation medium does not comprise a WNT surrogate. Preferably, the cell culture differentiation medium does not comprise EGF.

The in vitro method of the invention is performed in a cell culture medium which favors the growth and differentiation of the conjunctival stem-cells and/or the limbal stem-cells.

In a particular embodiment of the in vitro method, the culturing of the conjunctival stem-cells or of the limbal stem-cells in a culture medium comprises culturing in a cell expansion culture medium followed by culturing in a cell differentiation culture medium.

In another particular embodiment of the in vitro method according to the invention, the culturing in a culture medium, in particular in a cell differentiation culture medium, is for a period of time sufficient to allow formation of the conjunctiva organoids, preferably wherein the formed conjunctiva organoids comprises keratinocytes that express one or more of KRT-19, TP63, MUC1 and AQP5, and further comprises goblet cells.

More in particular, the formed conjunctiva organoids comprise cells that express KRT19, and one or more of TP63, MUC1, and AQP5.

Even more in particular, the formed conjunctiva organoids comprise basal cells that express KRT19, and TP63; and differentiated apical keratinocytes that express KRT19, and one or more of MUC1 and AQP5.

In another particular embodiment of the in vitro method according to the invention, the culturing in a culture medium, in particular in a cell differentiation culture medium, is for a period of time sufficient to allow formation of the limbal organoids, preferably wherein the formed limbal organoids comprises cells that express one or more of transcription factor Tumor protein p63 (TP63), Keratin-24 (KRT24), and PAX6 (Paired box protein Pax-6, also known as aniridia type II protein (AN2) or oculorhombin). In a preferred embodiment, the formed limbal organoids comprise cells that express KRT12. In some embodiments the cells do not or not substantially express MUC-1 (Mucin-1).

In another particular embodiment of the in vitro method according to the invention, the culturing in a culture medium, in particular in a cell differentiation culture medium, is for a period of time of at least 3 days. More in particular for a period selected from 3 days to 6 weeks, from 3 days to 5 weeks, from 3 days to 4 weeks, from 3 days to 2 weeks, and from 3 days to 1 week. In another more particular embodiment for a period selected from 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, and 42 days. In a particular embodiment in which short time is available, the culturing in a culture medium, in particular in a cell differentiation culture medium, is 3-4 days.

In a more particular embodiment of the in vitro method, the culturing of the conjunctival stem-cells and/or the limbal stem-cells in a culture medium comprises culturing in a cell expansion culture medium from at least 3 days, more in particular for at least 10 days, or a period selected from the ones previously indicated for the expansion medium, more in particular for a period selected from 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, and 56 days, or at least such periods; followed by culturing in a cell differentiation culture medium for at least 3 days, in particular for a period selected the ones previously indicated for the differentiation medium, more in particular selected from 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, and 42 days, or at least such periods. This particular sequence and timings of the expansion and differentiation culture media is particularly adequate to obtain conjunctiva organoids comprising differentiated keratinocytes and tuft cells and also goblet cells both at apical locations in relation to a support or an extracellular matrix (e.g., fibrin or basement membrane extract), or comprise limbal organoids as described herein.

The conditions that allow the conjunctiva stem-cells and/or the limbal stem-cells, in particular adult human conjunctival stem-cells and/or the adult human limbal stem-cells to form, respectively, a conjunctiva organoid or a limbal organoid, comprise the culturing in a medium that comprises an inhibitor of TGF-β signaling pathway; and inhibitor of BMP; and an agonist of the Wnt pathway, either in a cell culture expansion and/or differentiation medium.

In a particular embodiment of the in vitro method of the invention, the inhibitor of the TGF-β signaling pathway is a compound selected from the group consisting of A83-01 (CAS No.: 909910-43-6), SB-431542 (CAS No.: 301836-41-9), SB-505124 (CAS NO.: CAS 694433-59-5), SB-525334 (CAS No.: 356559-20-1), LY 364947 (CAS No.: 396129-53-6), SD-208 (CAS No.: 627536-09-8), SJN 2511 (CAS No.: 2319939-07-4), and combinations thereof, in particular it is A83-01 (CAS No.: 909910-43-6). Other suitable compounds are known to the skilled person.

In another particular embodiment of the in vitro method of the first aspect, the inhibitor of the BMP is a compound selected from the group consisting of Noggin (human protein Uniprot Nr. P97466), chordin, follistatin, gremlin, twisted grastulation (tsg), short gastrulation (sog), dorsomorphin, LDN193189 (CAS No.: 1062368-24-4), and combinations thereof. Other suitable compounds are known to the skilled person.

In another particular embodiment of the in vitro method of the first aspect, the agonist of the Wnt pathway is selected from the group consisting of R-spondin; a Wnt protein; a Wnt surrogate; a ROCK inhibitor and combinations thereof. Other suitable compounds are known to the skilled person.

In a more particular embodiment R-spondin is selected from the group consisting of in particular selected from R-spondin 1, R-spondin 2, R-spondin 3, and R-spondin 4, or a combination thereof. More in particular is R-spondin 1. Other suitable compounds are known to the skilled person.

In a more particular embodiment, the Wnt protein is selected from the compound CHIR99021 (CAS No.: 252917-06-9) or the compound iCRT3 (2-[[[2-(4-ethylphenyl)-5-methyl-4-oxazolyl]methyl]thio]-N-(2-phenylethyl) acetamide. Other suitable compounds are known to the skilled person.

Wnt surrogates are know to the skilled person, and include for example, WNT Surrogate-Fc Fusion Proteins, for example provided by IPAtherapeutics, and described by Miao et al (Cell Stem-cell (2020) Volume 27, Issue 5, Pages 840-851.e6. doi: 10.1016/j.stem.2020.07.020). Other suitable compounds are known to the skilled person.

The ROCK inhibitor is, in a particular embodiment, the compound Y-27632 (CAS No.: 146986-50-7). Other suitable compounds are known to the skilled person.

In a particular embodiment of the in vitro method according to the invention, once the conjunctiva organoids and/or limbal organoids are formed, it further comprises dissociating cells that are comprised in the organoid and subsequently culturing one or more of the dissociated cells in an air-liquid interface culture.

In a more particular embodiment, the in vitro method comprises the steps of:
(a) providing a container comprising a first chamber and a second chamber, said first and second chambers separated by a porous membrane;
(b) seeding of cells dissociated from the conjunctiva organoid and/or the limbal organoid on one side of the porous membrane, preferably in the first chamber, wherein said porous membrane is preferably coated first with an extracellular matrix (ECM) composition comprising one or more of collagen, laminin, and basement membrane extract, and the cells are seeded on this ECM;
(c) providing culture medium to the first and/or to the second chamber such that the seeded cells on the porous membrane are submerged in the culture medium;
(d) allowing the seeded cells to expand on the porous membrane for a period of time sufficient to obtain a substantially confluent layer of cells; and
(e) removing the culture medium from the chamber that includes the layer of confluent cells, preferably the first chamber, and allowing the cells to be in contact with air for period of time sufficient to obtain, respectively, a conjunctiva cell culture comprising multiple layers of cells, wherein the cell layers proximal to the porous membrane comprise basal keratinocytes that express KRT-19, and TP63 and wherein the cell layers distal to the porous membrane comprise apical keratinocytes that express MUC1, and goblet cells that express MUC5AC, or, a limbal cell culture comprising multiple layers of cells, comprising cells that express one or more of transcription factor Tumor protein p63 (TP63), Keratin-24 (KRT24), and PAX6 (Paired box protein Pax-6, also known as aniridia type II protein (AN2) or oculorhombin). In a preferred embodiment, the formed limbal organoids comprise cells that express KRT12, preferably wherein the culture medium is a cell expansion culture medium or a cell differentiation culture medium, more preferably a cell expansion culture medium as disclosed herein. In some embodiments the cells do not or not substantially express MUC-1 (Mucin-1), preferably wherein the culture medium is a cell expansion culture medium or a cell differentiation culture medium, more preferably a cell expansion culture medium as disclosed herein.

In another particular embodiment of the in vitro method, it comprises the steps of:
(a) providing a container comprising a first chamber and a second chamber, said first and second chambers separated by a porous membrane;
(b) seeding of conjunctival stem-cells, in particular from an isolated mammal conjunctiva, or seeding limbal stem-cells, in particular from isolated mammalian corneal limbus tissue and/or corneal tissue, on one side of the porous membrane, preferably in the first chamber, wherein said porous membrane is preferably coated first with an extracellular matrix (ECM) composition comprising one or more of collagen, laminin, and basement membrane extract, and the cells are seeded on this ECM;
(c) providing culture medium to the first and/or to the second chamber such that the seeded cells on the porous membrane are submerged in the culture medium;
(d) allowing the seeded cells to expand on the porous membrane for a period of time sufficient to obtain a substantially confluent layer of cells; and
(e) removing the culture medium from the chamber that includes the layer of confluent cells, preferably the first chamber, and allowing the cells to be in contact with air for period of time sufficient to obtain, respectively, a conjunctiva cell culture comprising multiple layers of cells, wherein the cell layers proximal to the porous membrane comprise basal keratinocytes that express KRT-19, and TP63 and wherein the cell layers distal to the porous membrane comprise apical keratinocytes that express MUC1, and goblet cells that express MUC5AC, or, a limbal cell culture comprising multiple layers of cells, comprising cells that express one or more of transcription factor Tumor protein p63 (TP63), Keratin-24 (KRT24), and PAX6 (Paired box protein Pax-6, also known as aniridia type II protein (AN2) or oculorhombin), preferably wherein the culture medium is a cell expansion culture medium or a cell differentiation culture medium, more preferably a cell expansion culture medium as disclosed herein. In a preferred embodiment, the formed limbal organoids comprise cells that express KRT12. In some embodiments the cells do not or not substantially express MUC-1 (Mucin-1). In a preferred embodiment, the formed conjunctiva organoids comprise tuft cells.

In a more particular embodiment of the in vitro method when it is carried out in an air-liquid interface, the conjunctiva cell culture and/or the limbal cell culture comprising multiple layers of cells comprises at least two layers, more in particular it comprises from 2 to 10 layers of cells.

When the method includes the culturing in an air-liquid interface culture, either departing from cells dissociated from the conjunctiva organoid, or from conjunctival stem-cells, in particular isolated from mammal conjunctiva, goblet cells are present in the obtained conjunctiva cell culture comprising multiple layers of cells as defined above.

The conjunctiva cell culture comprising multiple layers of cells or the limbal cell culture comprising multiple layers of cells can be detached from the chamber, preferably the first chamber, and be used directly as an implant (i.e., graft or transplant), for example of conjunctiva, or of corneal limbus tissue and/or corneal tissue (including corneal epithelium and/or limbal epithelium), for a subject in need thereof.

### Isolated Conjunctiva Organoids:

As previously indicated, another aspect of the invention is an isolated conjunctiva organoid, in particular a human conjunctiva organoid, comprising keratinocytes that express KRT19, and one or more of TP63, MUC1, and AQP5. In particular, the isolated conjunctiva organoid comprises basal cells that express KRT19, and TP63; and differentiated apical keratinocytes that express KRT19, and one or more of MUC1 and AQP5.

In another more particular embodiment of the isolated conjunctiva organoid of the invention, the isolated organoid, in particular human conjunctiva organoid, further comprises Goblet cells, preferably Goblet cells that express MUC5AC. In a more preferred embodiment, the conjunctiva further comprises tuft cells.

### Isolated Limbal Organoids:

As previously indicated, another aspect of the invention is an isolated limbal organoid, in particular a human limbal organoid, comprising cells expressing at least one of transcription factor Tumor protein p63 (TP63), Keratin-24 (KRT24), and PAX6 (Paired box protein Pax-6, also known as aniridia type II protein (AN2) or oculorhombin). In a preferred embodiment, the formed limbal organoids comprise cells that express KRT12. In some embodiments the cells do not or not substantially express MUC-1 (Mucin-1).

### Air-Liquid Interface Culture of Conjunctival Organoids:

As a result of the option of culturing in air-liquid interface culture, it is herewith provided an air-liquid interface culture of conjunctiva cells, preferably conjunctiva organoid cells, comprising:
i. a container comprising a first chamber and a second chamber, said first and second chambers separated by a porous membrane; and
ii. a multi-layer of conjunctiva cells disposed on at least one side of the porous membrane, said multi-layer comprising at least one layer of cells proximal to the porous membrane and comprising basal keratinocytes that express KRT-19 and TP63; and at least one layer of cells distal to the support and comprising apical keratinocytes that express KRT-19, MUC1 and AQP5, and goblet cells, and wherein the air-liquid interface culture optionally comprises a cell culture medium in at least one of the first and second chamber.

### Air-Liquid Interface Culture of Limbal Organoids:

As a result of the option of culturing in air-liquid interface culture, it is herewith provided an air-liquid interface culture of corneal epithelium and/or limbal epithelium cells, preferably limbal organoid cells, comprising:
i. a container comprising a first chamber and a second chamber, said first and second chambers separated by a porous membrane; and
ii. a multi-layer of limbal cells disposed on at least one side of the porous membrane, said multi-layer comprising cells expressing at least one of transcription factor Tumor protein p63 (TP63), Keratin-24 (KRT24), and PAX6 (Paired box protein Pax-6, also known as aniridia type II protein (AN2) or oculorhombin), and wherein the air-liquid interface culture optionally comprises a cell culture medium in at least one of the first and second chamber. In a preferred embodiment, the ALU culture cells comprise cells that express KRT12. In some embodiments the cells do not or not substantially express MUC-1 (Mucin-1).

### Conjunctiva or Limbal Implants, Transplants, or Grafts:

Another aspect of the invention is a conjunctiva implant or corneal limbus tissue and/or corneal tissue (including corneal epithelium and/or limbal epithelium) implant, respectively comprising, or consisting of, a conjunctiva organoid as defined above, or as obtained with a method as defined above, in particular a method comprising the air-liquid interface culture, or comprising or consisting of cells dissociated from said conjunctiva organoid; or comprising, or consisting of, a limbal organoid as defined above, or as obtained with a method as defined above, in particular a method comprising the air-liquid interface culture, or comprising or consisting of cells dissociated from said limbal organoid.

In a particular embodiment, the conjunctiva implant or corneal limbus tissue and/or corneal tissue implant comprises, respectively, the conjunctiva organoid as defined above or the limbal organoid as defined above, or as obtained with a method as defined above, in particular a method comprising the air-liquid interface culture, or the cells dissociated from said conjunctiva organoid or from said limbal organoid, and a physiologically acceptable material, preferably adhesive material.

The physiologically acceptable adhesive material is to be understood as a compound or composition which is biocompatible, in terms that it is non-toxic for the cells and in that it does not cause any adverse effect, such as an immune reaction, when in contact with the body tissues. Examples of these adhesive materials, also called bio-adhesives or biocompatible glues are, in a particular embodiment, selected from compositions comprising one or more of fibrin, laminin, albumin, and collagen, preferably fibrin. In a preferred embodiment, these adhesives are or derive from autologous materials from the subject which the implant is designed for. Examples of commercially available useful adhesives include Baxter Tisseel Fibrin (Baxter), Human collagen CC050 (Merck), and Laminin 1 (Human LAMA1/Laminin Alpha 1 Recombinant (His) Protein LS-G12401-100) (LS Bio).

Indeed, this physiologically acceptable adhesive material mimics or works as the extracellular matrix that confines the cells in a particular location. In even a more particular embodiment, the conjunctiva implant is a layered conjunctiva implant and comprises a layer of physiologically acceptable adhesive material; and one or more layers of the conjunctiva organoid, or of the cells from the dissociation of the organoid, or a conjunctiva cell culture comprising the multiple layers of cells from an air-liquid interface culture, disposed on the physiologically acceptable adhesive material layer. In even a more particular embodiment, the corneal limbus tissue and/or corneal tissue (including corneal epithelium and/or limbal epithelium) implant is a layered implant and comprises a layer of physiologically acceptable adhesive material; and one or more layers of the limbal organoid, or of the cells from the dissociation of the organoid, or a corneal limbus tissue and/or corneal tissue (including corneal epithelium and/or limbal epithelium) cell culture comprising the multiple layers of cells from an air-liquid interface culture, disposed on the physiologically acceptable adhesive material layer, as disclosed herein.

These implants with the physiologically acceptable adhesive material, are useful to accurately dispose the organoids or the cells obtained from their dissociation, or the layers of cells from an air-liquid interface culture, in a desired location of the body, without the risk of the lost within the eye.

### Conjunctiva Organoids as a Medicament:

As previously indicated, another aspect of the invention is an in vitro obtained conjunctiva organoid, or an in vitro obtained conjunctiva implant for use as a medicament, wherein the conjunctiva implant is as defined above, and wherein the in vitro obtained conjunctiva organoid is obtainable with a method as defined in any one of the first aspect or its embodiments, and/or wherein the in vitro obtained conjunctiva organoid is as defined in above.

In a particular embodiment of the in vitro obtained conjunctiva organoid, or of the in vitro obtained conjunctiva implant for use as a medicament, the use is in the prevention and/or treatment of a disease or disorder of the conjunctiva, in particular the human conjunctiva, or the use is in regenerative medicine.

In a particular embodiment, the in vitro obtained conjunctiva organoid, or the in vitro obtained conjunctiva implant is for use in an autologous cell therapy.

In another particular embodiment of the in vitro obtained conjunctiva organoid, or in vitro obtained conjunctiva implant for use as a medicament, it is for use in the prevention and/or treatment of one or more of actinic conjunctivitis, acute haemorrhagic conjunctivitis in Ghana, acute hemorrhagic conjunctivitis, alacrima, allergic conjunctivitis, chemical eye injury, chemosis, conjunctival concretion, conjunctival suffusion, conjunctivitis, conjunctivochalasis, dry eye syndrome, keratoconjunctivitis, ligneous conjunctivitis, mucus fishing syndrome, neonatal conjunctivitis, ocular melanosis, pinguecula, pseudopterygium, pterygium (eye), red eye, subconjunctival bleeding, superior limbic keratoconjunctivitis, symblepharon, vernal keratoconjunctivitis, eye cancer, in particular selected from eye melanoma, squamous cell carcinoma, conjunctival intraepithelial neoplasm, lymphoma, and retinoblastoma (a childhood cancer).

Some of these pathologies are the result of infections of the conjunctiva by pathogens. Thus, in also another particular embodiment of the in vitro obtained conjunctiva organoid, or in vitro obtained conjunctiva implant for use as a medicament, it is for use in the prevention and/or treatment of a conjunctival disease caused by a pathogen.

In a more particular embodiment, the pathogen are selected from virus, bacteria, and fungus.

This aspect and its embodiments can also be formulated as the use of an in vitro obtained conjunctiva organoid, or an in vitro obtained conjunctiva implant as above defined for the preparation of a medicament, in particular for the prevention and/or treatments of a disease or disorder of the conjunctiva, in particular the human conjunctiva.

The invention also relates to a method of treating a disease or disorder of the conjunctiva, in particular the human conjunctiva, comprising administering in a subject in need thereof, a therapeutically effective amount of an in vitro obtained conjunctiva organoid, or an in vitro obtained conjunctiva implant as above defined.

To be used as medicaments, the in vitro obtained conjunctiva organoid, or an in vitro obtained conjunctiva implant may be one of the integers of a pharmaceutical composition that comprises a therapeutically effective amount of the said organoid or implant, together with one or more pharmaceutically acceptable excipients or carriers.

The skilled person in the art will know the method to determine the said therapeutically effective amount and well as the possible pharmaceutically acceptable carriers or excipients.

### Limbal Organoids as a Medicament:

As previously indicated, another aspect of the invention is an in vitro obtained limbal organoid, or an in vitro obtained corneal limbus tissue and/or corneal tissue (including corneal epithelium and/or limbal epithelium) for use as a medicament, wherein the corneal limbus tissue and/or corneal tissue (including corneal epithelium and/or limbal epithelium) implant is as defined above, and wherein the in vitro obtained limbal organoid is obtainable with a method as defined herein, and/or wherein the in vitro obtained limbal organoid is as defined in above.

In a particular embodiment of the in vitro obtained limbal organoid, or of the in vitro obtained corneal limbus tissue and/or corneal tissue (including corneal epithelium and/or limbal epithelium) implant for use as a medicament, the use is in the prevention and/or treatment of a disease or disorder of corneal limbus tissue and/or corneal tissue (including corneal epithelium and/or limbal epithelium), in particular the human corneal limbus tissue and/or corneal tissue (including corneal epithelium and/or limbal epithelium), or the use is in regenerative medicine.

In a particular embodiment, the in vitro obtained limbal organoid, or the in vitro obtained implant is for use in an autologous cell therapy.

In another particular embodiment of the in vitro obtained limbal organoid, or in vitro obtained limbal/corneal epithelium implant for use as a medicament, it is for use in the prevention and/or treatment of one or more of limbal stem-cell deficiency, impaired corneal epithelial wound healing, chronic ocular surface inflammation, corneal neovascularization, corneal opacification, corneal recurrent epithelial erosion, corneal ulcers, corneal perforation, corneal epithelial neoplasm, dry eye syndrome, and/or for use in cultivated corneal epithelium and/or limbal epithelium transplantation therapy, ocular cancer therapy, aniridia therapy, and glaucoma therapy.

### Conjunctival or Limbal Organoids as In Vitro Model of, Respectively, Mammalian Conjunctival Epithelium or Mammalian Corneal Epithelium, Corneal Limbus, or Cornea, and Uses Thereof:

Another aspect of the invention is the use of the conjunctiva organoids as defined above, or of the air-liquid interface culture as defined above, as an in vitro model of mammalian conjunctival epithelia, or in drug discovery screening, or in toxicity assays.

Another aspect of the invention is the use of the limbal organoids as defined above, or of the air-liquid interface culture as defined above, as an in vitro model of mammalian corneal limbus tissue and/or corneal tissue (including corneal epithelium and/or limbal epithelium), including mammalian corneal limbus tissue and/or corneal tissue (including corneal epithelium and/or limbal epithelium), or in drug discovery screening, or in toxicity assays.

Yet another aspect is a method for the screening of a candidate agent to modulate conjunctiva and/or to prevent and/or treat a disease or disorder of the conjunctiva, in particular human conjunctiva, comprising:
(a) providing a conjunctiva organoid as defined above, or a conjunctiva air-liquid interface culture as defined above;
(b) optionally, providing a condition or an agent to simulate the disease or disorder;
(c) providing a candidate agent;
(d) contacting the candidate agent with the organoid or the air-liquid interface culture under conditions that allow interaction of the candidate agent with the organoid or the air-liquid interface culture, optionally, prior, after, or simultaneously to step (b); and
(e) determining if the contacting with the candidate agent modulates conjunctiva, the conjunctiva organoid or conjunctiva air-liquid interface culture and/or prevents or reverts the simulated disorder or disease.

Yet another aspect is a method for the screening of a candidate agent to modulate corneal limbus tissue and/or corneal tissue (including corneal epithelium and/or limbal epithelium) and/or to prevent and/or treat a disease or disorder of corneal limbus tissue and/or corneal tissue (preferably corneal epithelium and/or limbal epithelium), in particular wherein the tissue is human, comprising:
(a) providing a limbal organoid as defined in above, or a corneal limbus tissue and/or corneal tissue air-liquid interface culture as defined above;
(b) optionally, providing a condition or an agent to simulate the disease or disorder;
(c) providing a candidate agent;
(d) contacting the candidate agent with the organoid or the corneal limbus tissue and/or corneal tissue air-liquid interface culture under conditions that allow interaction of the candidate agent with the organoid or the air-liquid interface culture, optionally, prior, after, or simultaneously to step (b); and
(e) determining if the contacting with the candidate agent modulates the limbal organoid or corneal limbus tissue and/or corneal tissue air-liquid interface culture and/or prevents or reverts the simulated disorder or disease.

In a particular embodiment of the method of screening, the condition or agent to simulate the disease or disorder is a microorganism, in particular a bacteria or a virus.

Inventors could check that viral infections could be reproduced in the model of conjunctival organoid or limbal organoid. This supposes an important contribution, since by now for example some of the viral conjunctivitis needed to be studied using cancer cell lines derived from other tissues (e.g., lung), which do not reproduce, obviously, the same structure of the conjunctival tissue.

In another particular embodiment of the method of screening, the condition or agent to simulate the disease or disorder is the exposition of the organoid or of the air-liquid interface culture to desiccating stress, in particular for simulating dry eye. The desiccating stress to a cell culture can be applied according to the conventional techniques known by the skilled person in the art.

In another particular embodiment of the method of screening of a candidate agent to modulate conjunctiva, an agent is tested to determine if it can penetrate the conjunctiva epithelium, or if it can modulate the permeability of the conjunctiva. This particular method of screening, preferably carried out an air-liquid interface culture as defined above, allows to screen candidates able to reach the ocular tissues of the posterior or anterior chamber of the eye, which are, per se candidates or drugs to treat pathologies involving other ocular tissues, or which are compounds that aid other compounds can reach these other tissues by administration through the conjunctiva. For instance, glaucoma drugs that should reduce intra-ocular pressure are applied on the eye surface and must cross the conjunctival epithelium. The screening method of the invention, performed with the organoids or air-liquid interface culture also herewith disclosed, allows for the selection of candidates useful to reduce intra-ocular pressure and able to cross through the conjunctiva, but also it allows for the selection of candidates that will allow known glaucoma drugs to reach the corresponding ocular tissue through the conjunctiva. In the same manner, there is provided for the method of screening wherein an agent is tested to determine if it can penetrate or cross corneal epithelium and/or limbal epithelium, or if it can modulate such tissue. This particular method of screening preferably carried out a corneal epithelium air-liquid interface culture as defined above, allows to screen candidates able to reach the interior parts of the tissue or can reach the area below the tissue.

### Cell Culture Mediums for the Obtention of the Conjunctival Organoids:

Another aspect of the invention is a cell culture medium, preferably a cell expansion culture medium, comprising a basal medium for mammal cells supplemented with an inhibitor of the transforming growth factor beta (TGF-β) signaling pathway, an inhibitor of the bone morphogenic protein (BMP), and an agonist of the Wnt pathway, and optionally supplemented with one or more of B27 supplement; N-acetylcysteine (NAC); a fibroblast growth factor (FGF), in particular selected from FGF10, FGF1, FGF7, FGF2 and combinations thereof; an epidermal growth factor (EGF); and an antibacterial and/or antifungal compound. In some preferred embodiments, the culture medium further comprises forskolin or FGF-10 or WNT surrogate, or forskolin and FGF-10, or, preferably, forskolin, FGF-10 and WNT surrogate.

In some more preferred embodiments, the culture medium further comprises (e.g. next to forskolin or FGF-10 or WNT surrogate, or forskolin and FGF-10, or, preferably, forskolin, FGF-10 and WNT surrogate), IGF1 or FGF7, or, in a preferred embodiment IGF1 and FGF7. In yet further embodiments, the culture medium further comprises IL-6, for example, in combination with IGF1 and FGF7, for example in combination with forskolin, FGF-10 and WNT surrogate (or Wnt agonist). In a preferred embodiment, the culture medium is free or substantially free of EGF. In a preferred embodiment, the culture medium is free or substantially free of B27 supplement.

The cell culture medium, preferably cell expansion culture medium according to the previous aspect, comprises a fibroblast growth factor (FGF), in particular selected from FGF10, FGF1, and combinations thereof; and that it is substantially free of EGF.

Another particular embodiment of the cell culture medium, preferably cell expansion culture medium according to the previous aspect and embodiment, it further comprises interleukins, in particular one or more of interleukin-4 (IL4) and interleukin-13 (IL-13). In another embodiment, the culture medium comprises IL-6.

As previously indicated, a cell culture medium, preferably cell expansion culture medium, comprising the interleukins allow to mimic inflammatory conditions, which can be used to study the conjunctiva under this challenge.

Moreover, the presence in the cell culture medium, preferably cell expansion culture medium, of the interleukins, preferably one or more of IL4 and IL13, do also enhances the expansion of the goblet cells and/or tuft cells.

Another aspect of the invention is a cell culture medium, preferably a cell differentiation culture medium comprising basal medium for mammal cells supplemented with an inhibitor of the transforming growth factor beta (TGF-β) signaling pathway, an inhibitor of the bone morphogenic protein (BMP), and an agonist of the Wnt pathway, and which medium is substantially free of EGF, is substantially free of any fibroblast growth factor, and is substantially free of B27 supplement.

The invention may, with respect to the conjunctival stem-cells, also be summarized by the following clauses:
1. An in vitro method for producing a conjunctiva organoid comprising the steps of:
   (a) providing conjunctival stem-cells;
   (b) culturing the conjunctival stem-cells in a culture medium, and under conditions suitable to form a conjunctiva organoid.
2. The in vitro method according to clause 1 wherein culturing the conjunctival stem-cells in a culture medium comprises culturing in a cell expansion culture medium, preferably wherein the cell expansion culture medium comprises an inhibitor of the transforming growth factor beta (TGF-β) signaling pathway, an inhibitor of the bone morphogenic protein (BMP), and/or an agonist of the Wnt pathway.
3. The in vitro method according to any one of the previous clauses wherein culturing in culture medium is for a period of time sufficient to allow formation of the conjunctiva organoids, preferably wherein the formed conjunctiva organoids comprise keratinocytes that express one or more of transcription factor Tumor protein p63 (TP63), Keratin-19 (KRT19), mucin short variant S1 (MUC1), and aquaporin-5 (AQP5).
4. The in vitro method according to any one of the previous clauses wherein culturing in culture medium is for a period of at least 3 days.
5. The in vitro method according to any one of the previous clauses wherein the conjunctiva is a mammalian, rodent or human conjunctiva, preferably a human conjunctiva.
6. The in vitro method according to any one of the previous clauses, wherein the conjunctival stem-cells are human conjunctival stem-cells, and, preferably, wherein the culture medium/cell expansion culture medium is substantially free of epidermal growth factor (EGF) and, optionally, comprises one or more of a cyclic AMP activator, in particular forskolin, and/or one or more fibroblast growth factor(s) (FGF), in particular selected from FGF1 and FGF10, and combinations thereof.
7. The in vitro method according to any one of the previous clauses, wherein the conjunctival stem-cells, preferably the human conjunctival stem-cells, expresses nerve growth factor receptor (NGFR) or are nerve growth factor receptor positive conjunctival stem-cells.
8. The in vitro method according to any one of the previous clauses, wherein culturing the conjunctival stem-cells in a culture medium comprises culturing in a cell differentiation culture medium, preferably wherein the cell differentiation culture medium comprises an inhibitor of the transforming growth factor beta (TGF-β) signaling pathway, an inhibitor of the bone morphogenic protein (BMP), and/or an agonist of the Wnt pathway, and even more preferably, wherein the cell differentiation culture medium is further substantially free of EGF, fibroblast growth factor, and/or B27 supplement.
9. The in vitro method according to any one of the previous clauses, wherein culturing the conjunctival stem-cells in a culture medium comprises culturing in a cell expansion culture medium followed by culturing in a cell differentiation culture medium.
10. The in vitro method according to any one of the previous clauses, preferably any one of clauses 8-9; wherein culturing in culture medium, in particular in cell differentiation culture medium, is for a period of time sufficient to allow formation of the conjunctiva organoids, preferably wherein the formed conjunctiva organoids comprises keratinocytes that express one or more of KRT-19, TP63, MUC1 and AQP5, and further comprises goblet cells.
11. The in vitro method according to any one of the previous clauses, wherein the inhibitor of the transforming growth factor beta (TGF-β) signaling pathway is a compound selected from the group consisting of A83-01 (CAS No.: 909910-43-6), SB-431542 (CAS No.: 301836-41-9), SB-505124 (CAS NO.: CAS 694433-59-5), SB-525334 (CAS No.: 356559-20-1), LY 364947 (CAS No.: 396129-53-6), SD-208 (CAS No.: 627536-09-8), SJN 2511 (CAS No.: 2319939-07-4), and combinations thereof, in particular it is A83-01 (CAS No.: 909910-43-6).
12. The in vitro method according to any one of the previous clauses, wherein the inhibitor of the bone morphogenic protein (BMP) is a compound selected from the group consisting of Noggin (human protein Uniprot Nr. P97466), chordin, follistatin, gremlin, twisted gastrulation (tsg), short gastrulation (sog), dorsomorphin, LDN193189 (CAS No.: 1062368-24-4), and combinations thereof.
13. The in vitro method according to any one of the previous clauses, wherein the agonist of the Wnt pathway is selected from the group consisting of R-spondin 1 a Wnt protein, a Wnt surrogate, a ROCK inhibitor, and combinations thereof.
14. The in vitro method according to any one of the previous clauses, which further comprises dissociating cells that are comprised in the conjunctiva organoid and subsequently culturing one or more of the dissociated cells in an air-liquid interface culture.
15. The in vitro method according to clause 14, comprising the steps of:
   (a) providing a container comprising a first chamber and a second chamber, said first and second chambers separated by a porous membrane;
   (b) seeding of cells dissociated from the conjunctiva organoid on one side of the porous membrane, preferably in the first chamber;
   (c) providing culture medium to the first and/or to the second chamber such that the seeded cells on the porous membrane are submerged in the culture medium
   (d) allowing the seeded cells to expand on the porous membrane for a period of time sufficient to obtain a substantially confluent layer of cells; and
   (e) removing the culture medium from the chamber that includes the layer of confluent cells, preferably the first chamber, and allowing the cells to be in contact with air for period of time sufficient to obtain a conjunctiva cell culture comprising multiple layers of cells, wherein the cell layers proximal to the porous membrane comprise basal keratinocytes that express KRT19 and TP63 and wherein the cell layers distal to the porous membrane comprise apical keratinocytes that express MUC1, and goblet cells, in particular that express MUC5AC, preferably wherein the culture medium is a cell expansion culture medium or a cell differentiation culture medium, more preferably a cell expansion culture medium.
16. An isolated conjunctiva organoid, in particular a human conjunctiva organoid, comprising keratinocytes that express KRT19, and one or more of TP63, MUC1, and AQP5.
17. The isolated organoid, in particular human conjunctiva organoid, according to clause 16, which further comprises Goblet cells, preferably Goblet cells that express MUC5AC.
18. An isolated mammal conjunctiva organoid obtainable by a method as defined in any of clauses 1-15.
19. An air-liquid interface culture of conjunctiva cells, preferably conjunctiva organoid cells, comprising:
I. a container comprising a first chamber and a second chamber, said first and second chambers separated by a porous membrane; and
II. a multi-layer of conjunctiva cells disposed on at least one side of the porous membrane, said multi-layer comprising at least one layer of cells proximal to the porous membrane and comprising basal keratinocytes that express KRT-19 and TP63; and at least one layer of cells distal to the support and comprising apical keratinocytes that express KRT-19, MUC1 and AQP5, and goblet cells, and wherein the air-liquid interface culture optionally comprises a cell culture medium in at least one of the first and second chamber.
20. A conjunctiva implant, comprising, or consisting of, a conjunctiva organoid as defined in any one of clauses 16-18, or as obtained with a method as defined in any one of clauses 1-15, or comprising of consisting of cells dissociated from said conjunctiva organoid.
21. An in vitro obtained conjunctiva organoid, or an in vitro obtained conjunctiva implant for use as a medicament, wherein the conjunctiva implant is as defined in clause 20, and wherein the in vitro obtained conjunctiva organoid is obtainable with a method as defined in any one of clauses 1-15, and/or wherein the in vitro obtained conjunctiva organoid is as defined in any of clauses 16-18.
22. The in vitro obtained conjunctiva organoid, or in vitro obtained conjunctiva implant for use as a medicament according to clause 21, wherein the use is in the prevention and/or treatment of a disease or disorder of the conjunctiva, in particular the human conjunctiva, or the use is in regenerative medicine.
23. Use of the organoids as defined in any of clauses 16-18, or of the air-liquid interface culture as defined in clause 19, as an in vitro model of mammalian conjunctival epithelia, or in drug discovery screening, or in toxicity assays.
24. A method for the screening of a candidate agent to modulate conjunctiva and/or to prevent and/or treat a disease or disorder of the conjunctiva, in particular human conjunctiva, comprising:
(a) providing an organoid as defined in any of clauses 16-18, or an air-liquid interface culture as defined in clause 19;
(b) optionally, providing a condition or an agent to simulate the disease or disorder;
(c) providing a candidate agent;
(d) contacting the candidate agent with the organoid or the air-liquid interface culture under conditions that allow interaction of the candidate agent with the organoid or the air-liquid interface culture, optionally, prior, after, or simultaneously to step (b); and
(e) determining if the contacting with the candidate agent modulates conjunctiva and/or prevents or reverts the simulated disorder or disease.
25. The method of screening according to clause 24, wherein the condition or agent to simulate the disease or disorder is a microorganism, in particular a bacteria or a virus.
26. A cell culture medium, preferably a cell expansion culture medium, comprising a basal medium for mammal cells supplemented with an inhibitor of the transforming growth factor beta (TGF-β) signaling pathway, an inhibitor of the bone morphogenic protein (BMP), and an agonist of the Wnt pathway, and optionally supplemented with one or more of B27 supplement; N-acetylcysteine (NAC); a fibroblast growth factor (FGF), in particular selected from FGF10, FGF1, and combinations thereof; an epidermal growth factor (EGF); and an antibacterial and/or antifungal compound.
27. The cell culture medium, preferably cell expansion culture medium according to clause 26, which comprises a fibroblast growth factor (FGF), in particular selected from FGF10, FGF1, and combinations thereof; and that it is substantially free of EGF.
28. A cell culture medium, preferably a cell differentiation culture medium comprising basal medium for mammal cells supplemented with an inhibitor of the transforming growth factor beta (TGF-β) signaling pathway, an inhibitor of the bone morphogenic protein (BMP), and an agonist of the Wnt pathway, and which medium is substantially free of EGF, is substantially free of any fibroblast growth factor, and is substantially free of B27 supplement.

The invention may, with respect to the limbal stem-cells, also be summarized by the following clauses:
1. An in vitro method for producing a limbal organoid comprising the steps of:
(a) providing limbal stem-cells;
(b) culturing the limbal stem-cells in a culture medium, and under conditions suitable to form a limbal organoid.
2. The in vitro method according to clause 1 wherein culturing the limbal stem-cells in a culture medium comprises culturing in a cell expansion culture medium, preferably wherein the cell expansion culture medium comprises an inhibitor of the transforming growth factor beta (TGF-β) signaling pathway, an inhibitor of the bone morphogenic protein (BMP), and/or an agonist of the Wnt pathway.
3. The in vitro method according to any one of the previous clauses wherein culturing in culture medium is for a period of time sufficient to allow formation of the limbal organoids, preferably wherein the formed limbal organoids comprise cells that express one or more, preferably all of transcription factor Tumor protein p63 (TP63), Keratin-24 (KRT24), and PAX6 (Paired box protein Pax-6, also known as aniridia type II protein (AN2) or oculorhombin), preferably wherein the limbal organoid also comprises cells that express KRT12.
4. The in vitro method according to any one of the previous clauses wherein culturing in culture medium is for a period of at least 3 days.
5. The in vitro method according to any one of the previous clauses wherein the limbal cells are mammalian, rodent or human cells, preferably human cells.
6. The in vitro method according to any one of the previous clauses, wherein the limbal stem-cells are human limbal stem-cells, and, preferably, wherein the culture medium/cell expansion culture medium is substantially free of epidermal growth factor (EGF) and, optionally, comprises one or more of a cyclic AMP activator, in particular forskolin, and/or one or more fibroblast growth factor(s) (FGF), in particular selected from FGF1 and FGF10, and combinations thereof.
7. The in vitro method according to any one of the previous clauses, wherein the limbal stem-cells are human limbal stem-cells.
8. The in vitro method according to any one of the previous clauses, wherein culturing the limbal stem-cells in a culture medium comprises culturing in a cell differentiation culture medium, preferably wherein the cell differentiation culture medium comprises an inhibitor of the transforming growth factor beta (TGF-β) signaling pathway, an inhibitor of the bone morphogenic protein (BMP), and/or an agonist of the Wnt pathway, and even more preferably, wherein the cell differentiation culture medium is further substantially free of EGF, fibroblast growth factor, and/or B27 supplement.
9. The in vitro method according to any one of the previous clauses, wherein culturing the limbal stem-cells in a culture medium comprises culturing in a cell expansion culture medium followed by culturing in a cell differentiation culture medium.
10. The in vitro method according to any one of the previous clauses, preferably any one of clauses 8-9; wherein culturing in culture medium, in particular in cell differentiation culture medium, is for a period of time sufficient to allow formation of the limbal organoids, preferably wherein the formed limbal organoids comprises cells that express one or more, preferably all, of transcription factor Tumor protein p63 (TP63), Keratin-24 (KRT24), and PAX6 (Paired box protein Pax-6, also known as aniridia type II protein (AN2) or oculorhombin), preferably wherein the limbal organoid also comprises cells that express KRT12.
11. The in vitro method according to any one of the previous clauses, wherein the inhibitor of the transforming growth factor beta (TGF-β) signaling pathway is a compound selected from the group consisting of A83-01 (CAS No.: 909910-43-6), SB-431542 (CAS No.: 301836-41-9), SB-505124 (CAS NO.: CAS 694433-59-5), SB-525334 (CAS No.: 356559-20-1), LY 364947 (CAS No.: 396129-53-6), SD-208 (CAS No.: 627536-09-8), SJN 2511 (CAS No.: 2319939-07-4), and combinations thereof, in particular it is A83-01 (CAS No.: 909910-43-6).
12. The in vitro method according to any one of the previous clauses, wherein the inhibitor of the bone morphogenic protein (BMP) is a compound selected from the group consisting of Noggin (human protein Uniprot Nr. P97466), chordin, follistatin, gremlin, twisted gastrulation (tsg), short gastrulation (sog), dorsomorphin, LDN193189 (CAS No.: 1062368-24-4), and combinations thereof.
13. The in vitro method according to any one of the previous clauses, wherein the agonist of the Wnt pathway is selected from the group consisting of R-spondin 1 a Wnt protein, a Wnt surrogate, a ROCK inhibitor, and combinations thereof.
14. The in vitro method according to any one of the previous clauses, which further comprises dissociating cells that are comprised in the limbal organoid and subsequently culturing one or more of the dissociated cells in an air-liquid interface culture.
15. The in vitro method according to clause 14, comprising the steps of:
(a) providing a container comprising a first chamber and a second chamber, said first and second chambers separated by a porous membrane;
(b) seeding of cells dissociated from the limbal organoid on one side of the porous membrane, preferably in the first chamber;
(c) providing culture medium to the first and/or to the second chamber such that the seeded cells on the porous membrane are submerged in the culture medium
(d) allowing the seeded cells to expand on the porous membrane for a period of time sufficient to obtain a substantially confluent layer of cells; and
(e) removing the culture medium from the chamber that includes the layer of confluent cells, preferably the first chamber, and allowing the cells to be in contact with air for period of time sufficient to obtain a limbal cell culture comprising multiple layers of cells, wherein the cells express at least one, preferably all of transcription factor Tumor protein p63 (TP63), Keratin-24 (KRT24), and PAX6 (Paired box protein Pax-6, also known as aniridia type II protein (AN2) or oculorhombin), preferably wherein the limbal organoid also comprises cells that express KRT12, preferably wherein the culture medium is a cell expansion culture medium or a cell differentiation culture medium, more preferably a cell expansion culture medium.
16. An isolated limbal organoid, in particular a human limbal organoid, comprising cells that express at least one, preferably all of, transcription factor Tumor protein p63 (TP63), Keratin-24 (KRT24), and PAX6 (Paired box protein Pax-6, also known as aniridia type II protein (AN2) or oculorhombin), preferably wherein the limbal organoid also comprises cells that express KRT12.
17. The limbal organoid according to clause 16 wherein the limbal organoid is a human limbal organoid.
18. An isolated mammal limbal organoid obtainable by a method as defined in any of clauses 1-15.
19. An air-liquid interface culture of corneal limbus tissue and/or corneal tissue, preferably corneal epithelium and/or limbal epithelium, preferably limbal organoid cells, comprising:
I. a container comprising a first chamber and a second chamber, said first and second chambers separated by a porous membrane; and
II. a multi-layer of corneal limbus tissue and/or corneal tissue cells disposed on at least one side of the porous membrane, said multi-layer comprising cells wherein the cells express at least one, preferably all of transcription factor Tumor protein p63 (TP63), Keratin-24 (KRT24), and PAX6 (Paired box protein Pax-6, also known as aniridia type II protein (AN2) or oculorhombin), preferably wherein the limbal organoid also comprises cells that express KRT12, and wherein the air-liquid interface culture optionally comprises a cell culture medium in at least one of the first and second chamber.
20. A corneal limbus tissue and/or corneal tissue, preferably corneal epithelium and/or limbal epithelium implant, comprising, or consisting of, a limbal organoid as defined in any one of clauses 16-18, or as obtained with a method as defined in any one of clauses 1-15, or comprising of consisting of cells dissociated from said limbal organoid.
21. An in vitro obtained limbal organoid, or an in vitro obtained corneal limbus tissue and/or corneal tissue, preferably corneal epithelium and/or limbal epithelium implant for use as a medicament, wherein the corneal limbus tissue and/or corneal tissue, preferably corneal epithelium and/or limbal epithelium, implant is as defined in clause 20, and wherein the in vitro obtained limbal organoid is obtainable with a method as defined in any one of clauses 1-15, and/or wherein the in vitro obtained limbal organoid is as defined in any of clauses 16-18.
22. The in vitro obtained limbal organoid, or in vitro obtained corneal limbus tissue and/or corneal tissue, preferably including corneal epithelium and/or limbal epithelium, implant for use as a medicament according to clause 21, wherein the use is in the prevention and/or treatment of a disease or disorder of corneal limbus tissue and/or corneal tissue, preferably corneal epithelium and/or limbal epithelium, in particular human corneal limbus tissue and/or corneal tissue, preferably corneal epithelium and/or limbal epithelium, or the use is in regenerative medicine.
23. Use of the limbal organoids as defined in any of clauses 16-18, or of the corneal limbus tissue and/or corneal tissue air-liquid interface culture as defined in clause 19, as an in vitro model of mammalian corneal limbus tissue and/or corneal tissue (including corneal epithelium and/or limbal epithelium), or in drug discovery screening, or in toxicity assays.
24. A method for the screening of a candidate agent to modulate limbal organoids or corneal limbus tissue and/or corneal tissue (including corneal epithelium and/or limbal epithelium) and/or to prevent and/or treat a disease or disorder of corneal limbus tissue and/or corneal tissue (including corneal epithelium and/or limbal epithelium), comprising:
(a) providing a limbal organoid as defined in any of clauses 16-18, or a corneal limbus tissue and/or corneal tissue air-liquid interface culture as defined in clause 19;
(b) optionally, providing a condition or an agent to simulate the disease or disorder;
(c) providing a candidate agent;
(d) contacting the candidate agent with the organoid or the air-liquid interface culture under conditions that allow interaction of the candidate agent with the organoid or the air-liquid interface culture, optionally, prior, after, or simultaneously to step (b); and
(e) determining if the contacting with the candidate agent modulates the limbal organoid and/or the corneal limbus tissue and/or corneal tissue and/or prevents or reverts the simulated disorder or disease.
25. The method of screening according to clause 24, wherein the condition or agent to simulate the disease or disorder is a microorganism, in particular a bacteria or a virus.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein.

All references cited herein, including journal articles or abstracts, published or corresponding patent applications, patents, or any other references, are entirely incorporated by reference herein, including all data, tables, figures, and text presented in the cited references. Additionally, the entire contents of the references cited within the references cited herein are also entirely incorporated by references.

It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

It will be understood that all details, embodiments, and preferences discussed with respect to one aspect of embodiment of the invention is likewise applicable to any other aspect or embodiment of the invention and that there is therefore not need to detail all such details, embodiments, and preferences for all aspect separately.

Having now generally described the invention, the same will be more readily understood through reference to the following examples which is provided by way of illustration and is not intended to be limiting of the present invention. Further aspects and embodiments will be apparent to those skilled in the art.

### EXAMPLES

All the materials and methods that apply to the following examples are disclosed at the end of this section for the purpose of simplification.

### Example 1. Production of Mouse Conjunctiva Organoids

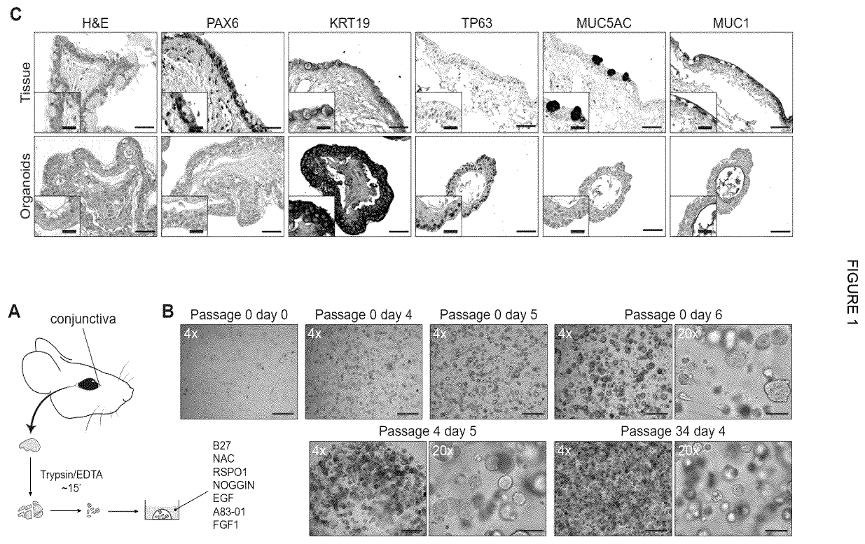

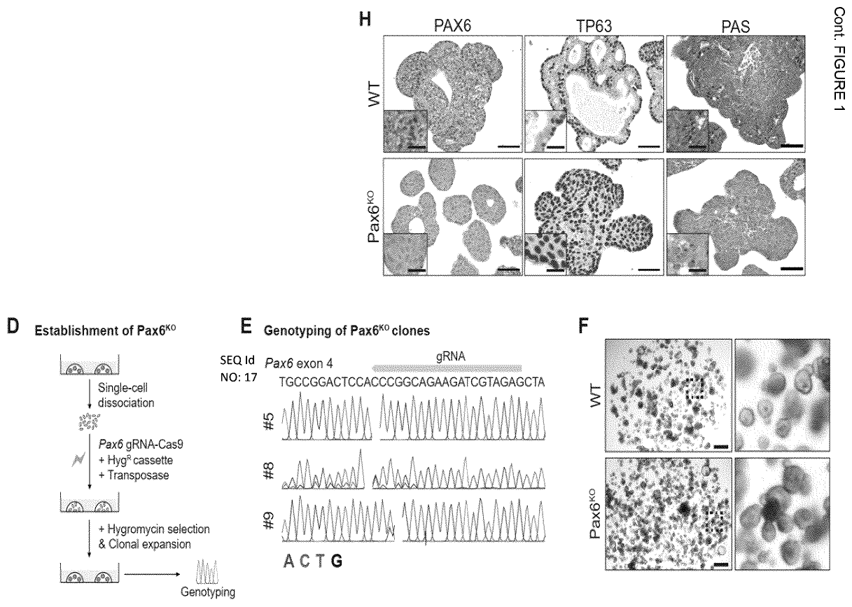

Wild-type mouse conjunctiva from eyelids and sclera was dissected, incubated in 0.25% Trypsin/EDTA for 5 minutes, and disrupted by pipetting before plating in Basement Membrane Extract (BME). The cultures were supplemented with a medium containing B27, N-acetylcysteine, Epidermal growth factor (EGF), Fibroblast growth factor-1 (FGF1), Noggin, R-spondin1, Tumor growth factor-β (TGFβ) inhibitor and Rho-kinase inhibitor (FIG. 1A). After 3-4 days, dense organoids appeared (FIG. 1B). Organoids were split on average every 7 days using Trypsin/EDTA and could be maintained for at least 34 passages (FIG. 1B). Organoids expressed the conjunctival marker Krt19, but not the corneal marker Krt12 (FIG. 1C). In addition, they retained the expression of the master transcription factor of eye development PAX6 (FIG. 1C). We identified cells that expressed the Tumor protein p63 (TP63+ cells) located basally in both mouse tissue and organoids and keratinocytes that expressed Mucin 1 (MUC1+ keratinocytes) in more apical locations (FIG. 1C). These expanding mouse conjunctival organoids did not contain Goblet cells that expressed Mucin 5AC (MUC5AC+ Goblet cells) (FIG. 1C), but they are equally useful for the study of conjunctiva.

### Pax6 is Essential for Conjunctival Differentiation:

Pax6 is the master regulator of eye development and maintains expression during adulthood. It has been reported that in certain conjunctival pathologies, such as pterygium and pinguecula, Pax6 expression is reduced. To assess the effect of Pax6 loss of function in conjunctival organoids, we mutated Pax6 with CRISPR/Cas9 (Pax6^{KO}, FIG. 1D-E). Pax6^{KO} organoids did not display overt morphological differences compared to their wild-type (WT) counterparts (FIG. 1F). Using bulk RNA sequencing, we found 709 downregulated genes and 454 upregulated genes in Pax6^{KO} compared to Pax6^{WT} organoids (fc>2 and p-adj<0.01, FIG. 1G). Pax6 mRNA was downregulated in Pax6^{KO} organoids, thereby confirming their genotype (FIG. 1G-H). Foxc2, another transcription factor key for ocular development, was also downregulated, implying that it acts downstream of Pax6 (FIG. 1G). Among the genes that were downregulated in Pax6KO organoids, we found several encoding secreted gene products, such as the antimicrobial peptides Ltf, Ctsz, Slpi, Pigr, Lcn2, Htra4, Serpina3n and Serpina9, the surfactant protein Sftpd, Fcgbp involved in maintaining gel structures, as well as the complement factors C3 and Cfh. We also noted that Toll-like receptors 2 and 4 (Tlr2 and Tlr4) were downregulated (FIG. 1G). Together, Pax6^{KO} triggered a decrease in defense-response genes. Conversely, genes that were upregulated in Pax6^{KO} organoids included stem-cell-related genes, such as the Wnt target Axin2 and the basal cell markers Trp63, Trp73, Krt5 and Krt14 (FIG. 1G). Immunohistochemistry confirmed that Pax6KO organoids showed increased expression of TP63 and decreased levels of PAS mucus/Goblet cell staining (FIG. 1H). In conclusion, Pax6 loss resulted in an undifferentiated basal-like phenotype, similar to that seen in pterygia.

### Example 2. Production of Human Conjunctiva Organoids

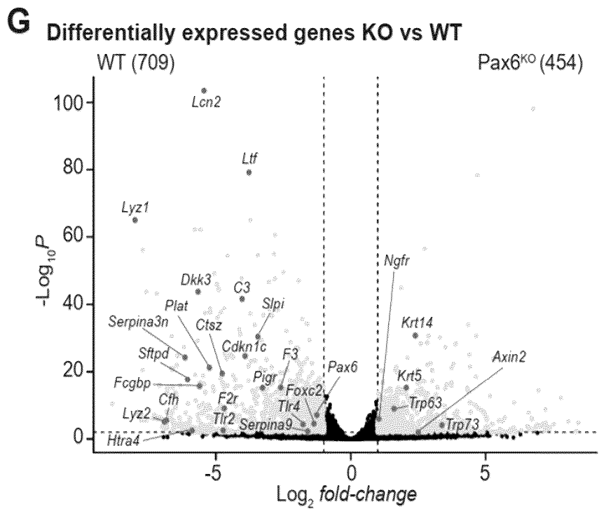

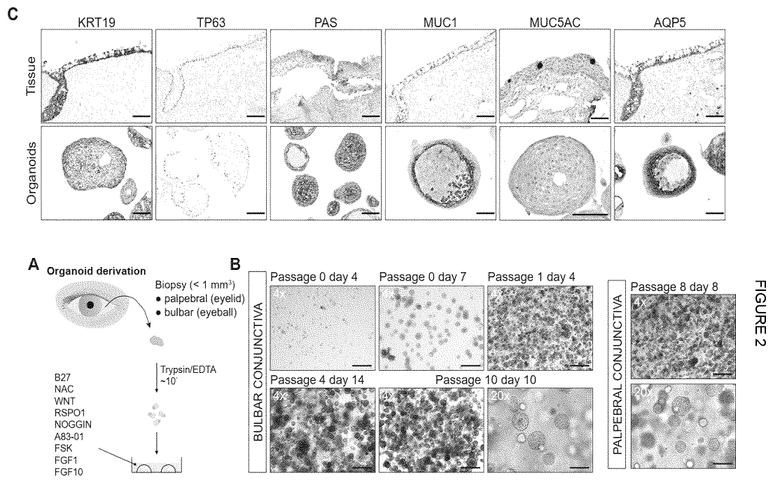

We then set out to establish human-derived conjunctiva organoids. For this, we obtained conjunctival biopsies from deceased donors and patients undergoing ocular surgeries. Biopsies were sampled from palpebral (eyelid) and bulbar (eyeball) conjunctiva and digested in 0.25% Trypsin/EDTA for 5 minutes. Single cells were then plated in BME in the mouse conjunctival medium supplemented with WNT surrogate, FGF10 and the cyclic AMP activator Forskolin (FSK). In addition, we removed EGF from the medium as it appeared to reduce the lifespan of the organoids (FIG. 2A). We obtained dense organoids from both bulbar and palpebral conjunctiva that could be enzymatically split every 9-14 days for up to 17 passages (FIG. 2B). Human conjunctival organoids retained expression of the conjunctival marker KRT19 in all cells (FIG. 2C). They also contained a basal layer of TP63+ cells, similar to the original tissue (FIG. 2C). Of note, the outside surface of organoids derived from any adult tissue contacts the BME and invariably represents the basal side of the corresponding epithelium. While organoids did not contain MUC5AC+ Goblet cells under these expansion conditions, they contained differentiated PAS+, MUC1+ and AQP5+ keratinocytes, morphologically very similar to tissue keratinocytes (FIG. 2C). Of note, AQP5 expression suggested that keratinocytes can secrete water and thus may participate in lubrication of the ocular surface in concert with the lacrimal gland.

Because human conjunctiva organoids did not contain Goblet cells under expansion conditions, we modified culture conditions. To do so, we removed the WNT surrogate, FGF1, FGF10, and B27 from the medium to create a “differentiation medium” (FIG. 2D). Upon exposure for 9 days to the differentiation medium, organoids became cystic and displayed increased expression of the Goblet cell transcription factor SPDEF and the secreted mucin gene MUC5AC (data not shown). We confirmed histologically that exposure to differentiation medium for 7 days reduced the number of TP63+ basal cells and proliferative cells, while increasing the numbers of MUC5AC+ cells (FIG. 2E). KRT19 remained expressed by all cells in differentiation medium, and MUC1 remained apical (FIG. 2E). Of note, MUC5AC staining was also detected in the lumen of some organoids, suggesting this mucin is secreted (FIG. 2E). Hence, differentiation of human conjunctiva organoids increased the expression of markers of differentiated cells, particularly Goblet cells, while reducing the number of basal cells.

### Example 3. Air-Liquid Interface Culture of Human Conjunctiva Organoids

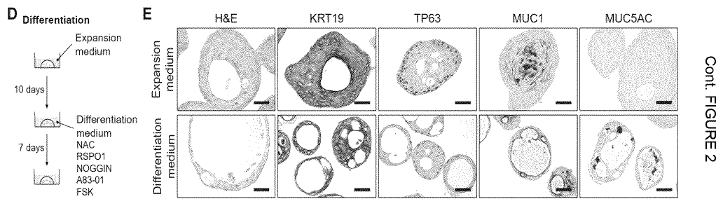

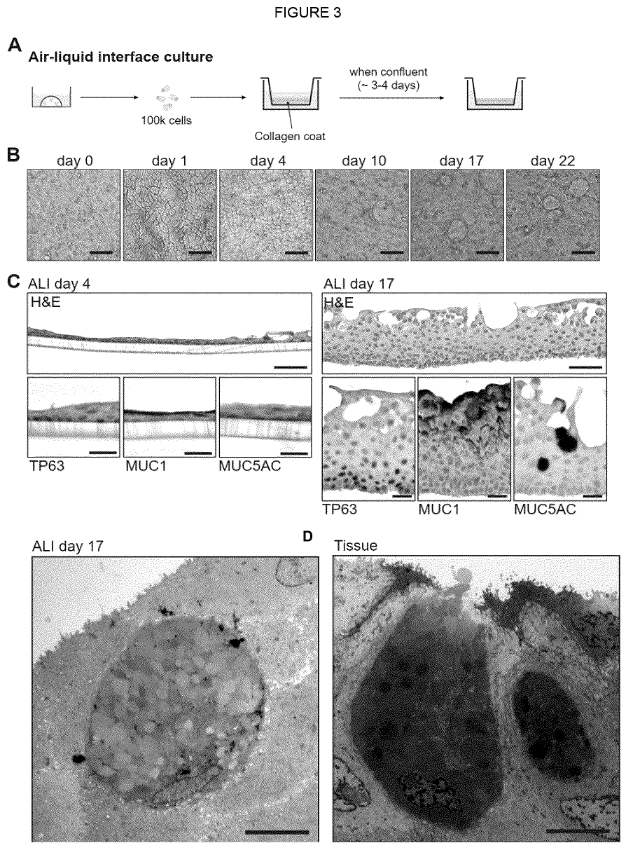

Air-liquid interface (ALI) cultures of epithelia that are normally exposed to air can promote near-native differentiation of cultured cells, such as has been shown for the upper airway. Thus, we seeded 100,000 conjunctival organoid cells on collagen-coated transwell plates (24-well) in human expansion medium. When cells reached confluency—which usually occurred within 3-4 days—, we removed the liquid in the upper chamber of the transwell to create an ALI (FIG. 3A). We followed the ALI cultures for up to 22 days, during which they remained confluent (FIG. 3B). At day 4 after shifting to ALI, the cultures were 2-3 cell layers-thick and displayed basal TP63+ cells and apical MUC1+ keratinocytes, but no MUC5AC+ Goblet cells (FIG. 3C). At day 17 after shifting to ALI, the cultures had become 5-10 cell layers-thick (FIG. 3C). TP63+ cells constituted the 2-3 basal-most layers, with small, densely packed cells (FIG. 3C). While early ALI cultures contained many KI67+ cells, the number of proliferative cells decreased over time to reach similar numbers as seen in primary tissue at 17-day post-ALI (data not shown) No substantial apoptosis was detected (data not shown). MUC1 was expressed in all cells, with gradual increase toward the apical surface (FIG. 3C).

Notably, 17-day-old ALI cultures contained MUC5AC+ cells (FIG. 3C). These MUC5AC+ cells resembled tissue Goblet cells: enlarged PAS+ vacuoles located at the surface or within the epithelium and containing MUC5AC+ vesicles. In addition, the ultrastructure of these cells, as demonstrated by transmission electron microscopy, was identical to tissue Goblet cells (FIG. 3D). We also found that differentiated ALI cultures expressed higher levels of the Goblet cell transcription factor SPDEF and of MUC5AC in comparison to their 3D organoid counterparts, while maintaining similar levels of the keratinocyte marker MUC16 and the antibacterial product WFDC2 (data not shown). Together, conjunctiva organoid-derived ALI cultures recapitulated the architecture, the cell type composition, and the functionality of human conjunctival tissue.

### Example 4. Single-Cell Characterization of Human Conjunctival Tissue and Cultures

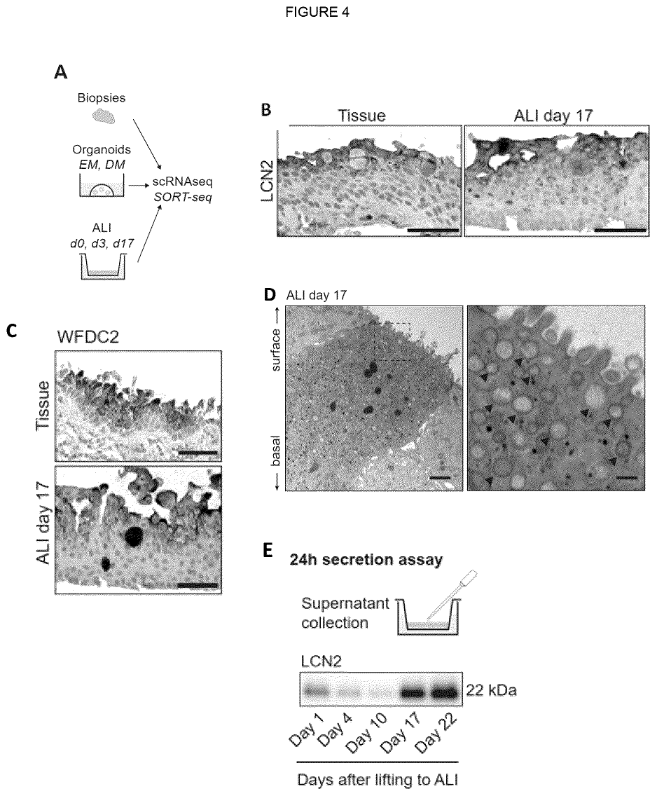

To compare our newly established culture systems directly to conjunctival tissue, we applied single-cell mRNA sequencing to (1) tissue biopsies (n=2), (2) organoids cultured in expansion or differentiation medium, and (3) ALI cultures 0, 3 and 17 days after shifting to ALI (FIG. 4A). As expected, non-epithelial cells only appeared in tissue biopsies (cluster 8). These included haemopoietic cells expressing PTPRC, CD3E, and CD4, melanocytes expressing MLANA, MITF and TYRBP1, and endothelial cells expressing PECAM1, VWF and TIE1 (Data not shown). Second, based on the expression of TP63, KRT5, and KRT14, we identified 5 clusters of basal cells (clusters 0, 3, 4, 5 and 7), among which one cluster was made up of MKI67+ proliferative basal cells mostly coming from early ALI cultures and tissue biopsies (cluster 7) (not shown). Lastly, we identified 3 clusters of differentiated keratinocytes based on the expression of MUC1 and MUC20 (data not shown). The dataset contained few Goblet cells, for two reasons: the fragile, large Goblet cells appeared highly susceptible to cell sorting-induced damage, and were relatively rare anyway, both in the bulbar biopsies and in the sequenced cultures. We identified 6 Goblet cells with high expression of MUC5AC, TFF1, TFF3 and SPDEF, originating from the tissue (n=3) and the organoids (n=3) (data not shown).

### Example 5. Characterization of Tissue Conjunctival Stem-Cells and Keratinocytes and Similarity Between Tissue and Organoid-Based Culture System. Functionality of Conjunctival Epithelium In Vitro

Since the conjunctival epithelium has not been extensively characterized at the single-cell level, we first focused on primary tissue cells. Using differential gene expression analysis (fold change >2, p-adjusted <0.01), we identified genes that were enriched in basal tissue cells (from clusters 0 and 7) and in differentiated keratinocytes from tissue (from cluster 1). Among the 23 genes enriched in basal cells, we identified several stem-cell markers (TP63, KRT5, KRT14), the proliferation marker MYC, COL17A1 (which was previously identified in stomach and skin stem-cells), as well as the basement membrane proteins LAMB1 and DST. In addition, basal cells were the main cells expressing the plasminogen activator PLAT, essential in the coagulation cascade, and the growth factors IGFBP6 and IGFBP7. Tissue-derived differentiated keratinocytes expressed genes involved in secretion, defence response and immune cell activation. Among these genes, keratinocytes expressed the complement factors C3, CFB and CFD, anti-microbial proteins SERPINA1, CST3, WFDC2, SERPINB1, S100A8, S100A9, SLPI and LCN2, the glycosylated adhesion proteins CEACAM5, CEACAM6 and CEACAM7, and the membrane-bound mucins MUC1, MUC4, MUC7, MUC15, MUC16, and MUC20. Conjunctival keratinocytes also appeared to participate in ocular retinol metabolism through the expression of ALDH1A3 and ADH1C, which are essential to maintain conjunctival and corneal epithelia in a non-keratinized state.

As a mucosal barrier component, we also found that conjunctival keratinocytes expressed the chemokines CXCL17 and IL18. CXCL17 is a chemokine known to be produced by other mucosal tissues, such as the gut and the lung, and is important in the recruitment of antigen-presenting cells, including monocytes macrophages and dendritic cells. Besides, CXCL17 is upregulated in tears of patients with autoimmune dry eye Sjögren's syndrome. Other mucosal chemokines involved in recruiting resident immune cells, including CCL25, CCL28 and CXCL14, were not expressed in conjunctival keratinocytes. While immune populations present in the mouse conjunctiva have recently been mapped, little is known about the role of the conjunctival epithelium in the recruitment of blood vessels, for instance, upon infection or allergy. We found that conjunctival epithelial cells, both stem-cells and keratinocytes, under homeostatic conditions expressed VEGFA and VEGFB, as well as low levels of VEGFC. Angiopoietins ANGPT1 and ANGPT2, as well as the VEGF signaling inhibitors THBS1 and THBS2 were not expressed by conjunctival epithelial tissue cells, in line with a previous report. Altogether, these data demonstrated the key role of conjunctival keratinocytes in protecting the ocular surface, by producing antimicrobial peptides and by interacting with the immune system through cytokine release.

### Similarity Between Tissue and Organoid-Based Culture Systems

Using this single-cell RNA sequencing dataset, we analyzed how cultured conjunctival cells compared to primary tissue cells. We thus compared the expression of key markers across culture conditions to tissue biopsies. As expected, conjunctival lineage markers KRT19 and PAX6 were expressed in all conditions (Data not shown). In addition, the expression of stem-cell markers KRT5, KRT14, TP63 and NGFR was broadly shared between organoid-derived cells and tissue biopsies (Data not shown). Stem-cell markers were most highly expressed in early ALI cultures and decreased over the course of differentiation, while differentiated markers (such as MUC1 and LCN2) were upregulated (Data not shown). Among markers upregulated over the course of in vitro differentiation (in organoids in DM and in ALI day 17), we found genes encoding membrane-bound mucins (MUC1, MUC4, MUC15, MUC16 and MUC20), anti-microbial peptides (LCN2, WFDC2, PIGR, SLPI), retinol metabolism enzymes (ADH7, RDH10, ALDH1A1, ALDH1A3 and ALDH3A1), complement factors (C3 and CFB) and the coagulation factor F3 (Data not shown). In addition, organoid-based cells retained expression of VEGFA and VEGFB, similar to that of the tissue (Data not shown). Expression levels of all the above-mentioned genes were very similar between differentiated organoid-based culture systems and tissue biopsies, implying that key tissue characteristics were recapitulated in vitro.

### Functionality of Conjunctival Epithelium:

Based on our single-cell RNA sequencing data, one function of the conjunctival epithelium would be to produce anti-microbial peptides. By antibody staining, we showed that the expression pattern of LCN2 and WFDC2 in 17-day-old ALI culture was identical to tissue samples (FIG. 4B-C). In addition, transmission electron microscopy showed the presence of medium size secretory vesicles in some, but not all, conjunctival keratinocytes of 17-day-old organoid-derived ALI cultures (FIG. 4D). We found that LCN2 was secreted in the supernatant in increasing quantities as the ALI cultures aged and differentiated (FIG. 4E). Hence, this data showed that conjunctival epithelium derived from organoids in vitro can secrete anti-microbial peptides.

### Example 6. Conjunctival Organoid from Single Cell

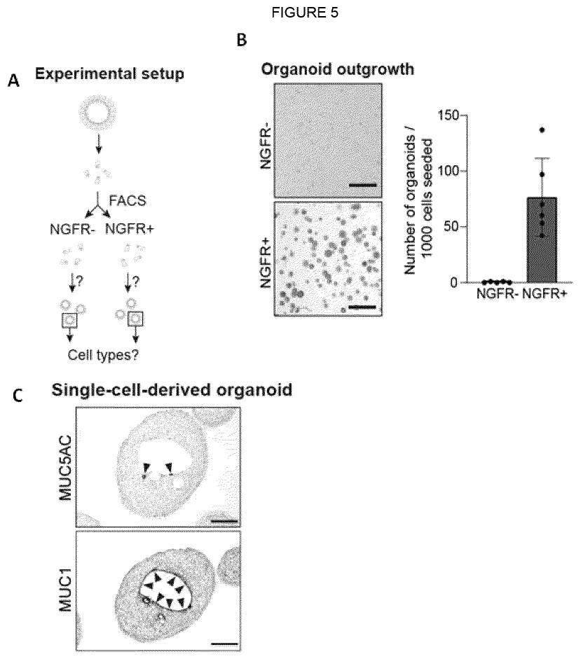

Next, we aimed to validate some of the findings generated by the single-cell RNA sequencing experiments. As we identified NGFR to be specifically expressed by basal cells, we assessed whether it represented a stem-cell marker in the conjunctival epithelium, as shown in other epithelia. We probed the organoid-forming capacity of sorted NGFR+ and NGFR-cells. Only NGFR+ cells were able to generate organoids. One question in the field is whether conjunctival stem-cells can generate both Goblet cells and keratinocytes. We identified both MUC1+ keratinocytes and MUC5AC+ Goblet cells in organoids derived from single NGFR+ cells. In sum, this experiment provided definitive evidence for NGFR+ cells as bipotent conjunctiva stem-cells (FIG. 5A, B, C).

### Example 7. Practical Application. Conjunctival ALI Cultures as Models of Conjunctival Viral Infections. Induction of Gene Expression

It has been hard to study conjunctival disorders in the lab due to a lack of long-term in vitro models. Therefore, we used our newly established conjunctival organoid technology to model viral conjunctivitis. Several viruses can infect the conjunctiva and the cornea, resulting in a sight-threatening condition called viral keratoconjunctivitis. Here, we focused on three viruses that can cause conjunctivitis: Herpes Simplex Virus 1 (HSV1), SARS-COV-2 and human Adenovirus 8 (hAdV8). HSV1 represents 5% of all conjunctivitis. Adenoviruses account for 90% of all conjunctivitis, of which hAdV8 is the most common strain worldwide. Patients with SARS-COV-2 present with conjunctivitis in 1% of cases. We first checked whether our human organoid models expressed the identified/potential viral entry receptors. HSV1 entry receptors (NECTIN1, NECTIN2 and HSPG2), SARS-COV-2 entry factors (ACE2 and TMPRSS2) and the proposed receptors for hAdV8 entry (ITGAV, ITGA3 and ITGB1) were expressed in all conjunctival culture models, especially in 17-day-old ALI cultures, where their expression was most similar to the tissue (data not shown).

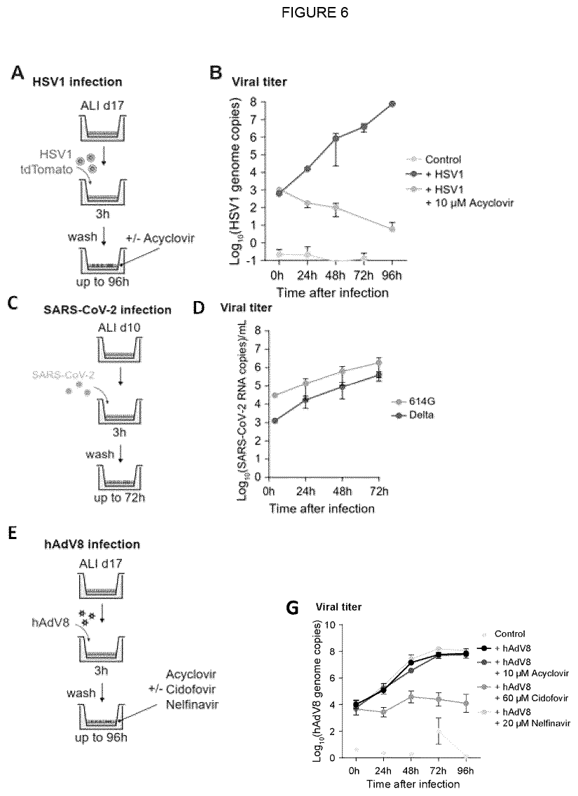

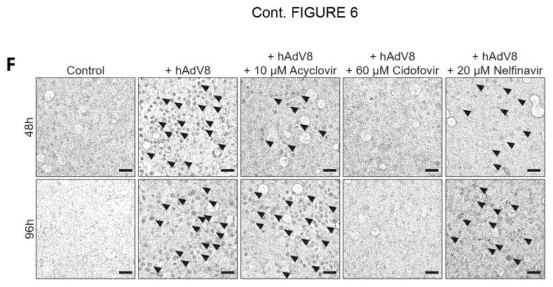

To model HSV1 infection, we exposed the apical surface of fully differentiated 17-day-old ALI cultures for 3 hours to an HSV1 strain whose capsid protein VP26 was tagged with tdTomato (hereafter called HSV1-tdTomato, FIG. 6A). HSV1 titrer increased by 5 logs over the course of 96 hours after initial incubation with HSV1, implying a productive infection of the conjunctival ALI culture with HSV1 (FIG. 6B). In addition, we observed that the tdTomato signal increased over time in HSV1-infected ALI cultures when imaged using an Auto-EVOS microscope (Thermo Fisher Scientific) (Data not shown). Interestingly, we noted tdTomato+ foci in cells, presumably where the virus is assembled in the nuclear envelope (Data not shown). tdTomato+ cells also displayed cytopathic effects (CPEs), including cell rounding and exclusion from the epithelial layer, resulting in large gaps in the ALI culture (Data not shown). Acyclovir, an inhibitor of HSV1 DNA polymerase, is commonly given to patients with HSV1-induced conjunctivitis. Indeed, the infection was blocked when ALI cultures were treated with 10 μM Acyclovir (FIG. 6A-B). Thus, organoid-derived ALI cultures support HSV1 infection and represent a human ocular surface model for HSV1 infection.

SARS-COV-2 is a respiratory virus that can also cause conjunctivitis. A recent study applied conjunctiva explants to study SARS-COV-2 infection. Yet, since a productive infection did not ensue, it was concluded that conjunctival epithelial cells resist productive SARS-COV-2 infection. To investigate whether SARS-COV-2 can directly infect the conjunctiva, we incubated SARS-COV-2 variants 614G and Delta with differentiated ALI cultures for 3 hours (FIG. 6C). Based on the concentration of RNA copies in the apical washes, both SARS-COV-2 variants replicated in conjunctival ALI cultures (FIG. 6D). Furthermore, the produced virus collected in ALI supernatant possessed secondary infectivity when added to Calu-3 cells (Data not shown). In addition, we identified cells positive for SARS-COV-2 nucleocapsid 72 hours after infection (Data not shown). Thus, infection by SARS-COV-2 of conjunctival epithelium can be studied in human organoids.

Adenoviruses are the leading cause of viral conjunctivitis. Yet, to date, no medication is available, partly because there is no model system for this type of ocular infection. An hAdV8 strain, isolated from a patient was provided by Dr. Nobuyo Yawata and Dr. Makoto Yawata (Kyushu University, Japan and NUS, Singapore) (see Yawata, N., and Yawata, M. 2022. Assessing the Response of Human NK Cell Subsets to Infection by Clinically Isolated Virus Strains. Methods Mol Biol 2463, 205-220. https://doi.org/10.1007/978-1-0716-2160-8_15). hAdV8 was added to 17-day-old conjunctival ALI cultures (FIG. 6E). Conjunctival ALI cultures were readily infected by hAdV8, as confirmed by the viral titre measured by qPCR (not shown). CPEs were visible starting from 48 hours after infection: infected cells darkened and were extruded from the epithelium. We then tested several antiviral drugs experimentally applied for adenoviral conjunctivitis (Acyclovir, Cidofovir and Nelfinavir) to test their efficacy in blocking the infection. We found that only Cidofovir could reverse adenoviral infection as shown by the decrease in viral titre and the absence of CPEs after up to 96 hours (FIG. 6F-G).

The cell-tropism of hAdV8 in the conjunctiva is unknown to date. To assess this, we studied which cell types were infected by hAdV8 in conjunctival ALI cultures. We found that both MUC5AC+ Goblet cells and MUC5AC-keratinocytes contained some hAdV8 capsid proteins (Data not shown), showing that hAdV8 can infect both Goblet cells and keratinocytes in the conjunctiva. Taken together, these data showed that organoid-based ALI cultures of the conjunctiva recapitulate viral infections.

### Example 8. Practical Application. Conjunctival Organoids for Autologous Cell Therapy

Replacement of the human conjunctiva using autologous cell therapy currently does not exist. To repair conjunctival damage currently, an autograft of healthy conjunctiva from another location in the patient's eye is harvested and used to cover the wound. The sclera underlying the donor site is left bare, and conjunctival scarring ensues. Consequently, no spare tissue is available for repeated procedures. Conjunctival organoid technology offers a unique potential to restore damaged conjunctiva as it requires less than 1 mm³ of tissue as starting material.

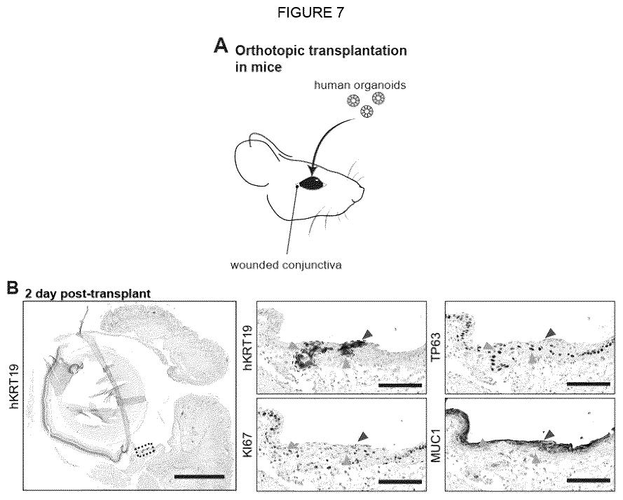

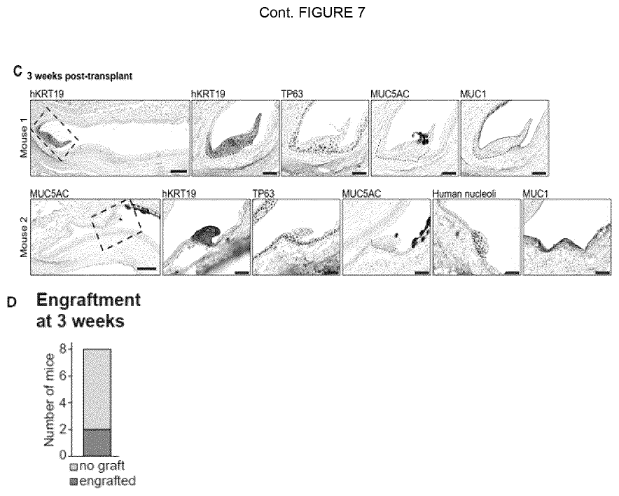

To test the engraftment capacity of human conjunctiva organoids, we transplanted these early after passaging in the bulbar and fornix conjunctiva of immunodeficient NSG mice that were previously mechanically wounded (FIG. 7A). Two days after transplantation, we found human cells engrafted in the mouse eye, as exemplified by human KRT19 staining (FIG. 7B). While only a few cells had engrafted and correctly integrated into the epithelium, we could already identify human cells that were located basally and apically (FIG. 7B). Basally located cells had retained expression of the stem-cell marker TP63, and some exhibited some KI67 positivity (FIG. 7B). Apically located cells, on the other hand, expressed the keratinocyte marker MUC1 (FIG. 7B). When we analyzed organoid engraftment after 3 weeks, we found human cells in 2 out of 8 mice (FIG. 7C-D). Importantly, in both engrafted mice, cells attached to the basement membrane and generated the 3 main cell conjunctival cell types: TP63+ basal cells, MUC1+ keratinocytes and MUC5AC+Goblet cells (FIG. 7C). While the surgical procedure is hard to perform on mouse eyes and requires improvement, this pilot experiment demonstrated that organoids receive adequate cues in vivo to trigger the pertinent differentiation cascades.

Precisely, to improve the surgical procedure, additional engineering of the organoids as a transplantable cell sheet was performed. Example 9 below illustrates this procedure.

### Example 9. Practical Application. Cell-Sheet of Conjunctival Organoids for Autologous Cell Therapy on a Fibrin Matrix

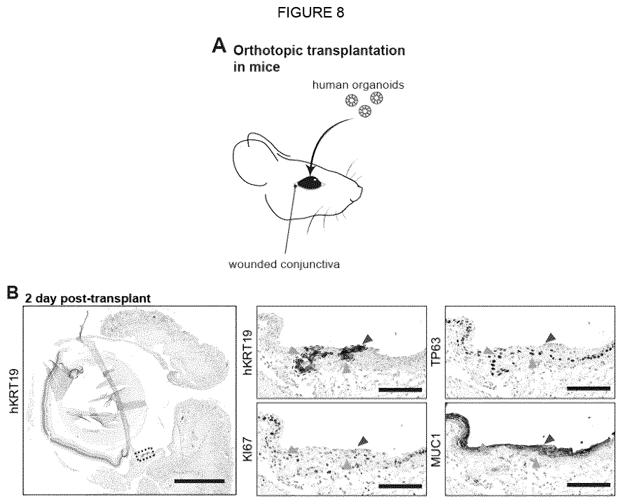

The conjunctiva is a flat polarized epithelium: stem-cells are located in contact with the basement membrane and differentiate towards the opposite side. We previously demonstrated that organoid-derived stem-cells could grow out on a coated 2D surface and differentiate into a near-native tissue architecture. Therefore, we went on to seed human organoid cells on a fibrin matrix to engineer a cell sheet easily amenable for transplantation in humans (FIG. 8A). Within 7 days, a fully confluent cell sheet could be generated on top of fibrin glue covering a 4 cm² surface (FIG. 8B). Importantly, cells grown on fibrin matrix could easily be lifted from the culture dish using forceps. After 7 days, stratification had begun and we identified both basal TP63+ cells and MUC1+ keratinocytes (FIG. 8B). In that setup, we did not find Goblet cells, probably because expansion medium was continuously provided apically and the time frame did not allow for final differentiation to occur. Yet, after 7 days, not all cells located basally expressed TP63 (FIG. 8B). In limbal stem-cell transplantations, the number of TP63+ cells in the final product is key to determine the outcome of the graft. Thus, we checked in earlier cultures seeded on fibrin the amount of basal and proliferative cells. We found by immunofluorescence analysis that when cells had just reached confluency, the conjunctival cell sheet was mostly comprised of TP63+ cells, some of which were also KI67+ (Data not shown). Therefore, we were able to engineer a cell sheet of the average size required for covering common conjunctival damages and easily amenable for transplantation in patients.

### Example 10. Organoid Outgrowth on a Collagen I as Extracellular Matrix

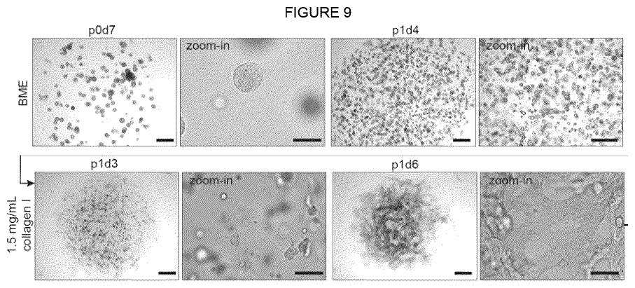

As performed in Example 2 human organoids were obtained from single cells derived from biopsies. The cells were expanded with the culture expansion medium previously disclosed in said Example 2. The cells were plated in collagen I (1.5 mg/ml). A comparative example was also performed with BME. Collagen could sustain organoid outgrowth (FIG. 9).

### MATERIALS AND METHODS RELATED TO THE EXAMPLES

### Mouse Organoids

Conjunctiva of surplus WT C57/BI6 female mice was dissected out and minced using a scalpel. No microdissection to remove the fibroblast layer was performed. The tissue suspension was incubated with 0.5% Trypsin-EDTA (Gibco) diluted 1:1 in Advanced DMEM/F12 (Gibco, final concentration 0.25%) for 10-15 minutes in a water bath at 37° C. The tissue suspension was vigorously pipetted up and down using a P1000 pipette every 5 minutes. Digestion was stopped when small epithelial fragments and/or single cells were obtained by adding 10 mL of Advanced DMEM/F12 (Gibco). Cells were then pelleted at 500 g for 5 minutes, washed a second time with 10 mL Advanced DMEM/F12 (Gibco) and pelleted again. The pellet was resuspended in about 100 μL per eye of Cultrex Pathclear Reduced Growth Factor Basement Membrane Extract (BME, 3533-001, Amsbio). BME was allowed to solidify for 25-30 minutes at 37° C. before adding expansion medium. Mouse expansion medium consisted in: Advanced DMEM/F12 (Gibco), 10 mmol/L HEPES (11560496, Gibco), GlutaMAX (11574466, Gibco), 100 U/mL Penicillin-Streptomycin (11548876, Gibco) (hereafter called AdDMEM+++), B27 Supplement (1X, 11530536, Gibco), 1.25 mM N-acetylcysteine (A9165, Sigma-Aldrich), 0.25% Noggin conditioned medium (U-Protein Express), 5% R-spondin 1 conditioned medium (produced as described in Pleguezuelos-Manzano, C., Puschhof, J., van den Brink, S., Geurts, V., Beumer, J., and Clevers, H. (2020). Establishment and Culture of Human Intestinal Organoids Derived from Adult Stem Cells. Curr Protoc Immunol 130, e106. 10.1002/cpim. 106.), 50 ng/ml EGF (AF-100-15, Peprotech), 100 ng/ml FGF1 (Peprotech), 3 μM A83-01 (2939, Tocris), 10 μM ROCK inhibitor Y-27632 (M1817, Abmole) and 100 mg/ml Primocin (ant-pm-1, Invivogen). Organoids were maintained in a humidified 37° C. incubator with 5% CO2. Every 7-10 days, organoids were dissociated to single cells using 0.5% Trypsin-EDTA (Gibco) diluted 1:1 in AdDMEM+++ similarly to organoid establishment and plated at a 1:6-1:8 ratio. When required, mouse expansion medium was changed for mouse differentiation medium 10 days after splitting. Mouse differentiation medium consisted in: AdDMEM+++, 1.25 mM N-acetylcysteine (A9165, Sigma-Aldrich), 0.25% Noggin conditioned medium (U-Protein Express), 5% R-spondin1 conditioned medium (produced as described in Pleguezuelos-Manzano, C., Puschhof, J., van den Brink, S., Geurts, V., Beumer, J., and Clevers, H. (2020). Establishment and Culture of Human Intestinal Organoids Derived from Adult Stem Cells. Curr Protoc Immunol 130, e106. 10.1002/cpim. 106.), 3 μM A83-01 (2939, Tocris), 10 μM ROCK inhibitor Y-27632 (M1817, Abmole) and 100 mg/mL Primocin (ant-pm-1, Invivogen). Mouse conjunctiva organoids were kept in differentiation medium for up to 9 days.

### Human Organoids

Human conjunctival samples were leftover material from patients undergoing eyelid surgeries at the University Medical Center Utrecht, the Netherlands (UMCU), from patients undergoing pterygium removal at the Maastricht University Medical Center, the Netherlands (MUMC+) or from donors at the ETB-BISLIFE cornea bank (Beverwijk, the Netherlands). This study was approved by the medical ethical committee (TCBio) of the UMCU as protocol 18-740, by the medical ethical committee of the MUMC+ under protocol METC 2021-2732 and by the ETB-BISLIFE donor bank and was in accordance with the Declaration of Helsinki and the Dutch law. Conjunctival samples of about 1 mm³ were kept cold in AdDMEM+++ supplemented with 100 mg/mL Primocin (ant-pm-1, Invivogen) until further processing (<4 hours). In the lab, human conjunctival samples were digested exactly like mouse samples. When needed, part of the sample was fixed for histological analyses (see after). The cell pellet was resuspended in BME and human expansion medium was added upon BME solidification. Human expansion medium consisted of mouse expansion medium supplemented with 0.15 nM Wnt Surrogate (U-Protein Express), 100 ng/ml FGF10 (100-26, Peprotech) and 1 μM Forskolin (1099, Tocris). As EGF was reducing the life span of the organoids (passage number), it was removed from the medium. Only single-cell mRNA sequencing of the organoids under expansion and differentiation was performed on organoids cultured in EGF-containing medium. Human conjunctival organoids were split every 10 days similarly to their mouse counterparts and plated at a 1:4 ratio. When needed, expansion medium was replaced for differentiation medium 10 days after splitting. Human differentiation medium consisted in AdDMEM+++, 1.25 mM N-acetylcysteine (A9165, Sigma-Aldrich), 0.25% Noggin conditioned medium (U-Protein Express), 5% R-spondin 1 conditioned medium, 3 μM A83-01 (2939, Tocris) and 10 μM ROCK inhibitor Y-27632 (M1817, Abmole). Human conjunctiva organoids were kept in differentiation medium for up to 11 days.

### Air-Liquid Interface Cultures

Human organoids (5-7 days after split) were harvested, trypsinized to single cells using 0.25% Trypsin/EDTA (ThermoFisher Scientific) for 5 minutes and 100,000 cells were seeded in 100 μL human expansion medium on 6.5 mm-wide transwells (Corning or Greiner). Human expansion medium was added to the lower compartment. After 3 to 4 days, when cells had reached confluency, medium from the upper compartment was removed to lift the cultures to air-liquid interface (ALI). The ALI cultures were maintained up to 22 days. The medium in the bottom compartment was changed every 3-4 days.

### RT-qPCR Analysis of Gene Expression

RNA was extracted from organoids contained in 100 μL of BME, from ALI cultures or primary tissue using the RNeasy Mini Kit (QIAGEN) and resuspended in 25 μL nuclease-free water (QIAGEN). Reverse transcription was performed on at least 500 ng RNA per condition with GoScript Reverse Transcriptase (A5003, Promega) and Random primers (C1181, Promega) according to the manufacturer's instructions. Quantitative PCR was performed with SYBR green (1725270, Bio-Rad) on a CFX384 Touch Real-Time PCR detection system (Bio-Rad). Primers used include:

MUC5AC_F2:
(SEQ ID NO: 1)
TCTGGAACGTGAGCATACCC,
MUC5AC_R2:
(SEQ ID NO: 2)
CGGCTCAAAGACCTTGCTCA,
MUC16_F1:
(SEQ ID NO: 3)
GCTACCACAGGTTCCAGTCC,
MUC16_R1:
(SEQ ID NO: 4)
CGACGGTTATAACTGCTGGTG,
SPDEF_F1:
(SEQ ID NO: 5)
CCAGTGGCCAACCTGAGTG,
SPDEF_R1:
(SEQ ID NO: 6)
TGGCGGCTGTGTCTGTTAG,
WFDC2_F1:
(SEQ ID NO: 7)
CAAGAGTGCGTCTCGGACAG,
WFDC2_R1:
(SEQ ID NO: 8)
TTCATCTGGCCAGGACACTG.

### Histology

Tissue was fixed in formalin for at least 2 hours. Organoids were dissociated from the BME by washing with 10 mL ice-cold AdDMEM+++ per 100 UL BME, followed by pelleting at 300×g for 5 minutes. Then, organoids and ALI cultures were also fixed in formalin for at least 2 hours. At that stage, the transwell membrane with cells on it was cut out from the transwell insert and further processed. Tissue, organoids and transwells were finally embedded in paraffin by performing serial incubations in EtOH 70%, EtOH 96%, EtOH 100%, Xylene and liquid paraffin. 4 μm-sections were made, hydrated, and subjected to hematoxylin and eosin (H&E) and PAS staining with the method widely known by the skilled person in the art, or to immunohistochemistry. For immunohistochemistry, antigen retrieval was performed according to the respective antibody manufacturer's instructions. Then, sections were blocked with 1% Bovine Serum Albumin (BSA, MP Biomedicals, 160069) in PBS. For staining, the following antibodies were used: TP63 (ab735, Abcam), PAX6 (Biolegend, 901301), KRT5 (clone AF138, Covance, 905501), KI67 (eBiosciences, 14-5698-82), MUC5AC (ThermoFisher Scientific, MA5-12175), MUC1 (Abcam, ab15481), AQP5 (Origene, TA307525), LTF (Millipore 07-685), LCN2 (R&D systems, AF1757), WFDC2 (LSBio, LS-C175346), mouse KRT19 (Cell Signalling Technology, 13092S), human KRT19 (Cell Signalling Technology, 4558S), and TFF3 (Atlas Antibodies, HPA035464). After overnight staining with the primary antibody at 4° C., sections were washed 3 times with PBS. If needed, sections were incubated with a secondary antibody rabbit anti-goat (Southern Biotech, 6160-01) for 1 hour and washed 3 times with PBS. Lastly, sections were incubated for one hour with BrightVision poly-HRT anti-rabbit (Agilent, K400311-2) or BrightVision poly-HRT anti-mouse (Agilent, K400111-2) and with 3,3′-diaminobenzidine (DAB) for 10 minutes. Finally, sections were dehydrated and mounted using Pertex®. Sections were imaged using a DM4000 optical microscope (Leica). Images were processed using the ImageJ software (FIJI).

### Immunofluorescence

First, organoids were retrieved from BME as described above. Organoids and ALI were fixed in formalin for 2-24 hours, permeabilized for 20 minutes in 0.2% Triton-X and blocked using 1% BSA and 0.2% Triton-X. Overnight staining was performed at 4° C. with the indicated antibody in 1% BSA and 0.2% Triton-X. Organoids and ALI were then washed 3 times in PBS and stained for one hour in the dark at room temperature while rotating with the appropriate secondary antibody: Alexa Fluor 488 donkey anti-rabbit (Thermo Fisher Scientific, A21206), Alexa Fluor 568 donkey anti-mouse (Thermo Fisher Scientific, A10037), Alexa Fluor 568 donkey anti-rabbit (Thermo Fisher Scientific, A10042) or Alexa Fluor 568 donkey anti-goat (Thermo Fisher Scientific, A11057). In some cases, Phalloidin-Atto 647N (Sigma-Aldrich, 65906) and DAPI (Sigma) were added to the mixture. After 3 washes of PBS and 1 wash of MilliQ, organoids and ALI on the transwell membrane were mounted in Prolong Gold antifade reagent with DAPI (P36935, Thermo Fisher Scientific) on a slide. Slides were imaged using either an SP8 or an SP8X confocal microscope (both Leica). Images were processed using ImageJ (FIJI).

### CRISPR/Cas9-Mediated Knock-Out

About 5 days after splitting (when the organoids were in the growth phase), they were dissociated into near single cell similar as for passaging. Half a plate was taken per gene to knock-out (~600 μL BME). The cell suspension was washed and pelleted at 500 g for 5 minutes. Then, organoids were resuspended in 80 μL BTXpress solution (45-0805, BTX) supplemented with 100 μM ROCK inhibitor Y-27632 (M1817, Abmole). 10 μg of pSpCas9 (BB)-2A-GFP containing Pax6 gRNA (Pax6_gRNA_F: CACCgCTCTACGATCTTCTGCCGGG (SEQ ID NO: 9) and Pax6_gRNA_R: AAACCCCGGCAGAAGATCGTAGAGc (SEQ ID NO: 10)) was cloned, 7.2 μg of hygromycin resistance containing transposon and 2.8 μg of transposase were added to the cell suspension. The suspension was transferred to an electroporation cuvette right before electroporation. Electroporation was performed with NEPA21. Immediately after, 400 μL of BTXpress supplemented with Y-27632 was added to electroporated cells and the cells were allowed to recover at room temperature for 30 minutes before plating. Importantly, a control without hygromycin transposon and transposase was also electroporated. This served as a selection control later on. After recovery, cells for Pax6 gRNA and control cells were plated at a similar density and expansion medium was added. When organoids had recovered from the electroporation and started to grow (about 3-5 days after electroporating), selection with Hygromycin (1:1000, Invivogen) was initiated. When all control cells had died (within <5 days), surviving clones were picked, dissociated, and clonally expanded. To check for Pax6 deletion, DNA from the clones was extracted using 50 μL QuickExtract™ DNA Extraction Solution 1.0 (QE09050, Lucigen) and PCR-mediated genotyping of the clones was performed using the following primers: Pax6_gen_F TCCAGTGGGCAGGTTCAAAT (SEQ ID NO: 11) and Pax6_gen_R AGGACGCTTAGAGTGGAGGGCC (SEQ ID NO: 12). Clones with out-of-frame deletions were kept for further analyses.

Bulk mRNA Sequencing

RNA was isolated from organoids, as done for qPCR analyses. Bulk mRNA sequencing was performed by Single Cell Discoveries (Utrecht, Netherlands). Briefly, polyA-enriched RNA was reverse transcribed, sequenced on an Illumina NextSeq500 and paired-end reads were mapped to the mouse (mm10) or human genome (hg19) available pipeline MapAndGo using the publicly (https://github.com/vertesy/TheCorvinas/blob/102b598cc8e3717c155c5c5ea974 488fe7992d96/Python/MapAndGo/Readme_MapAndGo.md) and the default settings for BWA-MEM. Analysis of bulk RNA-seq samples was performed in R. DESeq2 (version 1.26.0) (Love et al., 2014) was used to perform differential expression analysis between infected and naïve samples. clusterProfiler (version 3.14.0) (Yu et al., 2012) and ChIPpeakAnno (version 3.20.0) were applied to perform gene functional annotation of differentially expressed genes. When comparing gene expression levels, all samples were downsampled to the minimal depth (3.155 M transcripts per sample).

Single-Cell mRNA Sequencing

For single-cell mRNA sequencing of organoids, human conjunctival organoids cultured in expansion medium containing EGF (AF-100-15, Peprotech) for 17 days or in expansion medium containing EGF (AF-100-15, Peprotech) for 10 days and in differentiation medium for 7 days were dissociated into single cells, as for splitting. The cell suspension was then washed twice with 10 mL AdDMEM+++ and DAPI-negative cells were sorted in 384-well plates containing ERCC spike-ins (Agilent), RT primers and dNTPs (Promega) using a FACS Fusion (BD Biosciences).

For single-cell mRNA sequencing of tissue, human bulbar conjunctival biopsies were dissociated into single cells, similar to organoid establishment, and washed twice with AdDMEM+++. Cells were counted and stained with 5 μL per 10⁶ cells of the following antibodies for 30 minutes at 4° C. in the dark: Alexa Fluor 488 anti-human EPCAM (clone 9C4, 324210, Biolegend) and APC/Cy7 anti-Human CD45 (Clone HI30, 304014, Biolegend). Cells were washed and stained with DAPI immediately prior the sort. Living DAPI-negative epithelial cells (EPCAM-positive and CD45-negative) or ungated DAPI-negative cells were sorted into 384-well plates containing ERCC spike-ins (Agilent), RT primers and dNTPs (Promega) with a FACS Melody (BD Biosciences). Then, plates were processed according to the SORT-seq protocol, based on the Cel-Seq2 protocol. PolyA-enriched libraries were subjected to paired-end sequencing on an Illumina NextSeq500 platform. For analysis, fastq files were mapped to the human genome (hg19). Transcript counts from the different conditions were merged and inputted in the Seurat package v4.0. In total, we sequenced 1917 cells. After filtering out cells that had less than 1500 transcripts (n_features), more than 7500 transcripts (i.e. doublets) or fewer than 30% mitochondrial genes, and after reducing batch effects using SCtransform, we clustered 960 cells representing all 7 conditions. The following parameters were used to cluster the dataset:dims=1:11 and resolution=0.3. Cluster identity was determined based on the expression of marker genes. GOterm enrichment analyses were performed using the goseq package.

### Single-Cell Organoid Outgrowth

Human conjunctival organoids cultured in expansion medium for 7-11 days were dissociated into single-cells as described above. Cells were counted and stained with 5 μL per 10⁶ cells of PE anti-human NGFR (345106, Biolegend) for 30 minutes at 4° C. in the dark. Cells were then washed, resuspended in human expansion medium and strained (35 μm). DAPI was added immediately before the sort. 1000 living cells (DAPI-negative) NGFR-positive and NGFR-negative were sorted into human expansion medium, pelleted, and plated in 20 μL BME to follow organoid outgrowth. Pictures were taken on a brightfield Leica microscope and an AutoEvos (Thermo Fisher Scientific). The number of organoids growing out per condition was counted manually using ImageJ (FIJI) after 6 days. Single-cell clones were further expanded and subjected to histological analyses.

### Western Blot

24 hours before harvest, the upper compartment ALI cultures were washed with 100 μL of PBS to remove any previously secreted material. Then, 50 μL PBS was added on the apical surface of the ALI and the ALI was allowed to secrete products for 24 hours later, after which the supernatant was collected and frozen down until further processing. The same process was repeated at the indicated time points on the same ALI culture.

The collected supernatants were mixed with Laemmli buffer containing 100 mM DTT (Sigma) and boiled for 5 minutes. After spinning, supernatants were loaded on a 4-15% gradient gel (BioRad) and run at 100 V for 1 hour (LCN2 staining). Proteins were transferred to a nitrocellulose membrane with ice blocks at 100 V for 1 hour. The membrane was blocked using 1% BSA and stained overnight using LCN2 antibody (R&D systems, AF1757) in 1% BSA. The next day, membranes were washed 3 times with TBS containing 0.1% Tween (TBST, Sigma). LCN2-stained membranes were incubated with rabbit anti-goat HRP secondary antibody (Dako, P0449, 1:5000) for 1 hour in blocking buffer. After 3 washes with TBST, the membranes were incubated in ECL detection reagent (Thermo Fisher Scientific, 12393969) following the manufacturer's instructions. Membranes were imaged on a BioRad gel doc XR+.

### Herpes Simplex Virus 1 Infection

Herpes Simplex Virus 1 containing endogenously tagged VP16 capsid protein with tdTomato (HSV1-tdTomato) was used. For HSV1 infection of 17-day-old ALI cultures, HSV1-tdTomato virus was added apically to the ALI (in the transwell) in 50 μL PBS at an MOI of 0.1 for 3 hours. After 3 hours, the supernatant was removed and the transwell was washed 2 times with 150 μL PBS. Then, every 24 hour, the secreted virus was collected in 100 μL PBS (including at 0-hour post-infection). In brief, PBS was added to the transwell, pipetted up and down 3 times and collected after 15 minutes. The collected supernatant was then stored at −20° C. until further processing. If indicated, 10 μM acyclovir (PHR1254, Sigma-Aldrich) was added to the bottom compartment after incubating with the virus. In addition, ALI cultures were imaged daily to monitor viral progression using an Auto-EVOS microscope (Thermo Fisher Scientific). Viral titer quantification was performed as indicated below.

hAdV8 Infection

An hAdV8 strain isolated from a patient was used. Similar to HSV1 infection, hAdV8 at MOI 10 was added to the ALI culture in 50 μL PBS for 3 hours, before being washed away. Every 24 hours, the shed viruses were collected in 100 μL PBS and stored until further processing. If indicated, 10 μM Acyclovir (PHR1254, Sigma-Aldrich), 60 μM Cidofovir (C5874, Sigma-Aldrich) or 20 μM Nelfinavir (PZ0013, Sigma-Aldrich) was added to the bottom compartment after incubating with the virus. hAdV8-infected ALI cultures were kept for up to 4 days and, if required, fixed.

Determination of the Viral Titers by qPCR

At the time of processing of the pellets for organoids and supernatants for ALI cultures, DNA was extracted using Zymogen Quick-DNA Microprep kit (ZY-D3020, Zymogen). DNA was systematically resuspended in 15 μL nuclease-free water (QIAGEN). Viral DNA was then quantified by qPCR as follow: 5 μL of the DNA extract was mixed together with 5 μL of SYBR green (Bio-Rad) containing HSV1 detection primers (HSV1-F: ATCAACTTCGACTGGCCCTT (SEQ ID NO: 13) and HSV1-R: CCGTACATGTCGATGTTCAC (SEQ ID NO: 14)) or hAdV8 detection primers (hAdV8-F: TTCCCCATGGCNCACAAYAC (SEQ ID NO: 15) and hAdV8-R: TGCCKRCTCATRGGCTGRAAGTT (SEQ ID NO: 16). The SYBR green was mixed with 10 μM primer mix at a ratio 10:1. Technical duplicates were assessed by qPCR. The following program was used for viral DNA amplification: 95° C. for 2 minutes, followed by 40 cycles of 98° C. for 15 seconds, 60° C. for 15 seconds and 72° C. for 15 seconds. For quantifying the viral DNA content in ALI supernatants, we amplified 179 bp of HSV1-tdTomato genome using HSV1-F and HSV1-R and 1004 bp of the hAdV8 genome using hAdV8-F and hAdV8-R. We cloned both in a pJet vector (Thermo Fisher Scientific) and used it to make a standard curve ranging from 2.104 to 2.109 copies of viral genome according to Addgene instructions (https://www.addgene.org/protocols/aav-titration-qpcr-using-sybr-green-technology/). Using this standard curve, we deduced how many viral genome copies were shed in the supernatant. Infection experiments were always conducted in technical triplicates and repeated in different organoid lines.

### SARS-COV-2 Production

Calu-3 cells were maintained in Opti-MEM I (1X)+GlutaMAX (Gibco), supplemented with 10% fetal calf serum, penicillin (100 IU/mL) and streptomycin (100 IU/mL) at 37° C. in a humidified CO2 incubator. Ancestral SARS-COV-2 (614G, isolate BavPat1/2020 EVAg Ref-SKU: 026V-03883) and the Delta variant (GenBank accession number: OM287123) were propagated on Calu-3 cells in AdDMEM+++, and sequence confirmed as described before (GeurtsvanKessel et al., 2022). Virus titrations were performed by plaque assay. All work with infectious SARS-COV-2 was performed in a Class II Biosafety Cabinet under BSL-3 conditions at Erasmus Medical Center.

### SARS-COV-2 Infection

For SARS-COV-2 infections of 17 to 30-day old ALI cultures, expansion medium was refreshed and cultures were washed twice with 200 μL AdDMEM+++ before inoculation from the apical side at a MOI of 0.1 in 200 μL AdDMEM+++ per well. Next, cells were incubated at 37° C. and 5% CO2 for 2 hours before washing the apical side 3 times in 200 μL AdDMEM+++. At the indicated timepoints, virus was collected from the cells by adding 200 μL AdDMEM+++ apically, incubating 10 minutes at 37° C. with 5% CO2, and storing the supernatant at −80° C. Prior to determining the virus titer, samples were centrifuged at 500×g for 5 minutes. Infectious virus titers were determined using by qRT-PCR and by plaque assay on Calu-3 cells.

### Fixed Immunofluorescence Microscopy of SARS-COV-2-Infected 2D Cultures

Cells were fixed in formalin, permeabilized in 70% ethanol, and blocked for 60 minutes in 10% normal goat serum in PBS (blocking buffer). Cells were incubated with primary antibody (Rabbit-anti-SARS-COV NP, Sino Biological; 1:1000) overnight at 4° C. in blocking buffer, washed with PBS, incubated with Alexa488-coupled secondary antibody (1:1000; Invitrogen) in blocking buffer for 2 hours at room temperature, washed with PBS, incubated for 20 minutes with Hoechst, incubated for 20 minutes in CruzFluor647-labeled phalloidin (Santa Cruz), washed with PBS, and mounted in Prolong Antifade (Invitrogen) mounting medium. Samples were imaged on an LSM700 confocal microscope using ZEN software (Zeiss).

### Transmission Electron Microscopy

Fresh tissue and ALI samples were fixed in fixation buffer (2% paraformaldehyde, 2.5% glutaraldehyde in 0.1 M phosphate buffer at pH 7.4) for 24 hours at 4° C. Then, samples were kept in wash buffer (0.1 M cacodylate) until further processing. Samples underwent an additional fixation with 1% osmium tetroxide and 1.5% potassium ferricyanide in wash buffer for 1 hour in the dark at 4° C. Further, the samples were dehydrated in EtOH 100% and infiltrated with Epon resin for 2 days. 20 Then, samples were embedded in Epon resin, which polymerized for 2 days at 60° C. Ultrathin sections were cut using an ultramicrotome (Leica Ultracut UCT) and mounted on Formvar-coated copper grids. Sections were stained with 2% uranyl acetate in water and lead citrate. Sections were imaged using a Tecnai T12 electron microscope and an Eagle 4 k*4 k CCD camera (Thermo Fisher Scientific). 25

### Orthotopic Transplantations in Mouse

Human bulbar conjunctiva organoids were expanded as described in the previous sections and split 4-5 days before the transplantation day. On the transplantation day, organoids were freed from BME using dispase. Organoids in their growth phase were resuspended in fibrin component 2 (TISSEEL, Baxter) and kept on ice until transplantation in mice. Immunodeficient NSG mice were sedated by intra-peritoneal injection of midazolam (10 μL/g body weight). Under a surgical microscope, the bottom bulbar conjunctiva of the right eye was excised using surgical scissors. Then, 5 μL of organoid suspension in fibrin component 2 (~100,000 cells) was placed on top of the wound. ~5 μL of fibrin component 1 was immediately added to induce fibrin polymerization. After the fibrin had polymerized, one drop of Fucithalmic ointment was added on the eye to prevent bacterial infections. The eyelids were stitched together to avoid blinking-related failure of the transplantation. To prevent pain, mice were injected with 0.1 mg/kg of buprenorphine on the day of the surgery and were provided with 0.06 mg/ml of carprofen in the drinking water for 2 days. Mice were sacrificed using CO2 inhalation 2 days after the surgery to assess initial engraftment of the organoids. The entire eye was dissected, fixed, and embedded in paraffin. Screening for engraftment of KRT19+ human cells was performed histologically. When human cells were detected, consecutive sections were stained for conjunctival markers including TP63, MUC1 and KI67 as described in the previous chapter. All mouse experiments were conducted under a project license granted by the Dutch government's Central Committee Animal Experimentation (CCD) and approved by the KNAW-Hubrecht Institute Animal Welfare Body.

### Data Analysis and Availability

The number of replicates and the statistical analysis performed is indicated in the figures or legends.

### Organoids Grown on Fibrin

First, a fibrin gel was prepared in a suspension plate immediately before seeding cells. Human conjunctival organoids 5 days after the previous split were dissociated to single cells using 0.25% Trypsin/EDTA. After filtering the cell suspension using a 40 μm filter (Greiner), 1.2×10⁶ cells were seeded per well of a 12 well plate in human expansion medium. After up to 7 days, cells grown on fibrin were fixed in formalin and subjected to histology and immunofluorescence as described in the previous sections.

### Organoid Derivation in Collagen

First, rat tail collagen I (ThermoFisher Scientific) was prepared according to the manufacturer's instructions to make a gel. Second, human conjunctival tissue was dissociated as previously described. Identical amounts of dissociated cells were seeded in BME and 1.5 collagen I side by side to compare organoid outgrowth. Splitting of organoids grown on collagen I was performed using 0.25% Trypsin/EDTA.

### Example 11. Engraftment of Human Conjunctival Organoids in a Rabbit Model

The conjunctiva (cells lining the sclera and inner surface of the eyelid) is essential for ocular lubrication and antibacterial defense. Damage can occur by mechanical or chemical injury, but also due to the treatment of premalignant pterygia or ocular surface squamous neoplasia, a group of diseases incorporating pre-cancerous and malignant epithelial lesions (conjunctival intraepithelial neoplasia, squamous cell carcinoma and its rare variant mucoepidermoid carcinoma). The defect can be repaired by an autograft, but the tissue source is limited and leads to donor-site scarring or is even unavailable. In this experiment pre-clinical evidence is obtained that shows that human conjunctival cells/grafts obtained with the method as disclosed herein are able to be used as implant, and therefore suitable for use in treatment of conjunctival epithelium damage.

Graft preparation and assessment was basically as follows. A small conjunctival biopsy (<1 mm²) is taken from a donor, which is then processed according to the methods disclosed herein where the stem-cells present in the conjunctival epithelium proliferate and efficiently form organoids. The organoids are grown in 3D conditions in basement membrane extract and cells are passaged on average every 10 days by dissociating and re-plating them in basement membrane extract.

For the preparation of the graft for transplantation, the cells are seeded on a fibrin gel approximately 3 to 7 days after they have been passaged (when they are still in their proliferation phase). Currently, around 2.5 million cells are seeded in a 30 mm dish containing a medical-grade fibrin gel. Approximately 4 days after seeding, the cells are confluent, and the grafts can be prepared. Using a 6 mm punch, we can generate around 6-8 grafts from one plate, which is then transplanted in rabbit conjunctiva in vivo. Our preliminary trials demonstrated that we need to optimize the seeding density.

Human conjunctival graft were prepared on fibrin as described above and the obtained grafts were transplanted into 20 rabbits for the 7-day follow-up group and another 14 rabbits for the 21-day follow-up group.

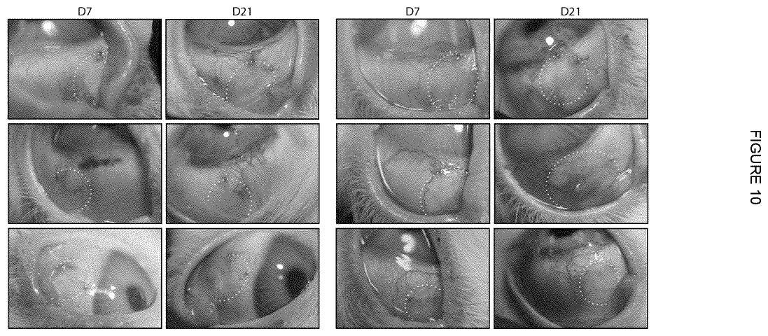

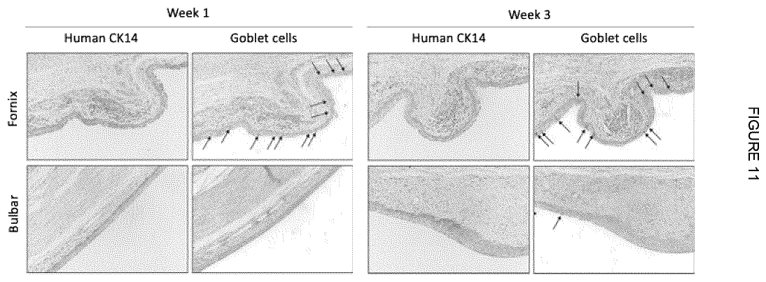

FIG. 10 shows the region of the defect (region where the implant is introduced) with a dashed white line (5 mm diameter). The sutures that marked the boundaries of the graft are clearly visible, which is an indication of how well the defect healed. Representative images of the histology at weeks 1 and 3 show the graft is populated with multiple layers of human epithelial cells. At week 1, ~100% of the graft is populated, whereas it is ~75% at week 3. The arrows in FIG. 11 indicate Goblet cells (Alcian blue), which are (as expected) more prevalent in the fornix (18 cells/mm) than the bulbar (9 cells/mm) conjunctiva. Multiple layers of human cells were observed, which is relevant for a regenerated ocular surface.

### Example 12. Production of Human Limbal Organoids

In this example we set out to establish human-derived limbal organoids The methodology followed equals that described for human conjunctival organoids above. The cornea constitutes the transparent layer of the anterior eye. This layer consists of extracellular matrix-secreting keratocytes organized perpendicularly to light trajectory covered by a non-keratinized stratified epithelium. This epithelium is renewed by stem-cells located around the cornea, in a region called the limbus. During homeostasis and after injury of the central cornea, these stem-cells divide and migrate centripetally to differentiate and replenish the tissue. If an injury occurs in the limbus area, this can lead to limbal stem-cell deficiency. In such a case, the limbal stem-cells, and ultimately the central cornea, become replaced by conjunctival tissue leading to blindness.

Because of this, both the cornea and the limbus have been the subject of intense investigations. Some culture models exist to study the cornea. These models are either limbal stem-cells expanded on mouse embryonic fibroblasts (2D structure) or induced pluripotent stem-cells (iPSC)-derived organoids (3D structure). Here, we establish and characterize organoids directly from limbal stem-cells.

For this, human limbal/corneal samples that were leftover material from donors at the ETB-BISLIFE cornea bank (Beverwijk, the Netherlands, n=7) were used. This study was in accordance with the Declaration of Helsinki and the Dutch law. As patient samples were anonymized, sex, gender, age, race, and other information were not recorded and is not available either for the organoid lines derived downstream of some of these samples.

The limbus region was cut out and the samples were minced and placed in 0.05% Trypsin/EDTA (ThermoFisher Scientific) in a water bath at 37° C. for 30 minutes, while shaking the tube regularly. The corneal pieces were allowed to settle before collecting the supernatant and placing it in a tube containing 10 ml of Advanced DMEM/F12 (ThermoFisher Scientific) supplemented with Pen/Strep (ThermoFisher Scientific), GlutaMAX (ThermoFisher Scientific) and HEPES (ThermoFisher Scientific) (thereafter called Ad+++). If cells were still attached to the corneal matrix, the above step was repeated a second time and dissociated cells were collected and pooled with the previous ones.

After pelleting the cells at 300×g for 5 minutes, these were resuspended in Basement Membrane Extract (BME, Cultrex). The droplets of BME containing corneal cells were then plated in a suspension plate (Greiner). When the BME was solidified, expansion medium was added. The human expansion medium consisted of Ad+++ supplemented with B27 Supplement (1X, 11530536, ThermoFisher scientific), 1.25 mM N-acetylcysteine (A9165, Sigma-Aldrich), 0.25% Noggin conditioned medium (U-Protein Express), 5% R-spondin 1 conditioned medium (produced as described in Pleguezuelos-Manzano, C., Puschhof, J., van den Brink, S., Geurts, V., Beumer, J., and Clevers, H. (2020). Establishment and Culture of Human Intestinal Organoids Derived from Adult Stem Cells. Curr Protoc Immunol 130, e106. 10.1002/cpim. 106.), 100 ng/ml FGF1 (Peprotech), 3 μM A83-01 (2939, Tocris), 10 μM ROCK inhibitor Y-27632 (M1817, Abmole), 0.15 nM Wnt Surrogate (U-Protein Express), 100 ng/ml FGF10 (100-26, Peprotech) and 1 μM Forskolin (1099, Tocris). As EGF was reducing the life span of the organoids (passage number), it was removed from the medium.

In additions organoids were grown in the (human) expansion medium defined above, supplemented with 100 ng/ml FGF-7 (Peprotech) and 100 ng/ml IGF-1 (X2 medium) or with 100 ng/mL FGF-7 (Peprotech), 100 ng/ml IGF-1 (Peprotech), and IL-6 (Peprotech) (X3 medium).

Organoids were split exactly like human conjunctiva organoids.

For two-dimensional culture on transwells, corneal organoids were seeded and maintained in 2D exactly like human conjunctiva organoids. If indicated, PBS was placed in the upper chamber of the transwell when cells had reached confluency.

Histological and immunofluorescence analyses were performed exactly like for human conjunctiva.

### Results

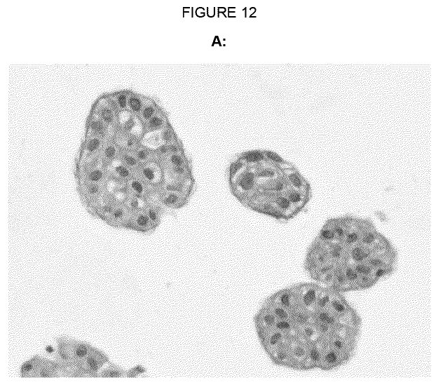

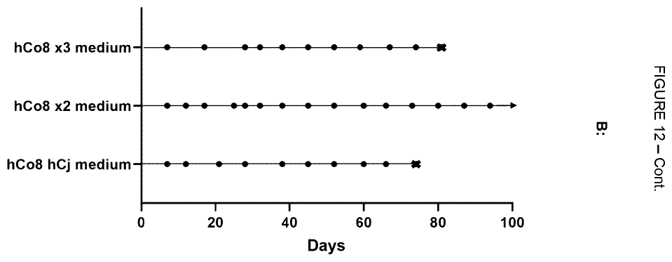

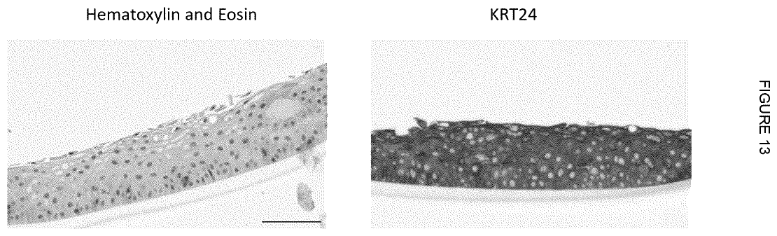

After dissecting the whole cornea or the limbus, the cells were dissociated using 0.05% Trypsin/EDTA and plated in a medium containing B27, N-acetylcysteine, WNT surrogate, R-spondin 1 conditioned medium, Noggin conditioned medium, TGFβ inhibitor A83-01, Forskolin (FSK), FGF1 and FGF10. Dense organoids grew out within 7 days and were then split every 7-14 days on average, at least for up to 3 months and 10 splits (passages) or more (X2 medium; at least 14 splits/passages), demonstrating that with the method according to the invention long-term (e.g., more than 10 passages, culturing of limbal (or in the case of conjunctiva: conjunctival) stem-cells and/or organoids is provided (See FIGS. 12A and B). Established limbal organoids expressed the stem-cell marker TP63 basally and the limbal stem-cell marker KRT24, as well as PAX6. Lastly, organoids grew similarly in the (human) expansion medium defined above, or in the X2 medium or X3 medium for the first 10 passages. The expansion medium further comprising IGF1 and/or FGF7 provided overall the best long-term outgrowth, allowing at least up to 14 passages or more.

We then characterized histologically the human limbal organoids as compared to limbus and central cornea tissue. The organoids according to the invention expressed the ocular surface transcription factor PAX6. Most cells expressed TP63, which was most similar to the limbal region of the tissue. KRT24, a marker for so-called “limbal superficial epithelium”, was most expressed apically in the organoids, while its expression was widespread throughout the limbus and cornea epithelial tissue. Lastly, no MUC1 was expressed in neither the organoids nor the tissue. Overall, the human organoids resembled histologically their corresponding tissue.

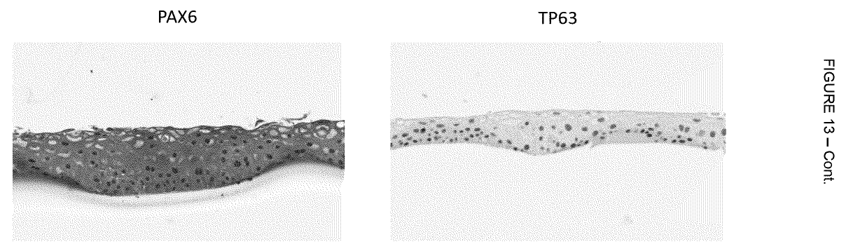

To recapitulate the architecture of the limbal tissue (corneal epithelium tissue), we set out to grow the organoids on a transwell in two dimensions (2D culture). Until confluency, both chambers of the transwell were filled with expansion medium. When the cells became confluent, either the upper chamber of the transwell was filled with PBS or all liquid was removed. We found that after about 7 days, the cells had stratified and harbored a basal layer of cells expressing the stem-cell marker TP63, as well as KRT24 and PAX 6 (FIG. 13). This architecture is very similar to that of the central corneal tissue.

Having now fully described this invention, it will be appreciated by those skilled in the art that the same can be performed within a wide range of equivalent parameters, concentrations, and conditions without departing from the spirit and scope of the invention and without undue experimentation.

Reference to known method steps, conventional methods steps, known methods or conventional methods is not in any way an admission that any aspect, description, or embodiment of the present invention is disclosed, taught, or suggested in the relevant art.

1. An in vitro method for producing a conjunctiva organoid or a limbal organoid comprising the steps of:
(a) providing conjunctival stem-cells and/or providing limbal stem-cells;
(b) culturing the conjunctival stem-cells and/or limbal stem-cells in a culture medium, and under conditions suitable to form a conjunctiva organoid or a limbal organoid.

2. The in vitro method according to claim 1, wherein culturing the conjunctival stem-cells or limbal stem-cells in a culture medium comprises culturing in a cell expansion culture medium, wherein the cell expansion culture medium comprises an inhibitor of the transforming growth factor beta (TGF-β) signaling pathway, an inhibitor of the bone morphogenic protein (BMP), and/or an agonist of the Wnt pathway.

3. The in vitro method according to claim 1, wherein culturing in culture medium is for a period of time sufficient to allow formation of the conjunctiva organoids, wherein the formed conjunctiva organoids comprise keratinocytes that express one or more of transcription factor Tumor protein p63 (TP63), Keratin-19 (KRT19), mucin short variant S1 (MUC1), and aquaporin-5 (AQP5), or to allow the formation of limbal organoids.

4. (canceled)

5. The in vitro method according to claim 1, wherein the conjunctiva and/or limbal organoid is a human conjunctiva or limbal organoid.

6. The in vitro method according to claim 1, wherein the conjunctival stem-cells or the limbal stem-cells are human conjunctival stem-cells or human limbal stem-cells, and wherein the culture medium/cell expansion culture medium is substantially free of epidermal growth factor (EGF) and comprises one or more of a cyclic AMP activator, and/or one or more fibroblast growth factor(s) (FGF).

7. The in vitro method according to claim 1, wherein the conjunctival stem-cells expresses nerve growth factor receptor (NGFR).

8. The in vitro method according to claim 1, wherein culturing the conjunctival stem-cells and/or the limbal stem-cells in a culture medium comprises culturing in a cell differentiation culture medium, wherein the cell differentiation culture medium comprises an inhibitor of the transforming growth factor beta (TGF-β) signaling pathway, an inhibitor of the bone morphogenic protein (BMP), and/or an agonist of the Wnt pathway, and wherein the cell differentiation culture medium is further substantially free of EGF, fibroblast growth factor, and/or B27 supplement.

9. The in vitro method according to claim 1, wherein culturing the conjunctival stem-cells and/or the limbal stem-cells in a culture medium comprises culturing in a cell expansion culture medium followed by culturing in a cell differentiation culture medium for a period of time sufficient to allow formation of the conjunctiva organoids and/or limbal organoids, wherein the formed conjunctiva organoids comprises keratinocytes that express one or more of KRT-19, TP63, MUC1 and AQP5, and further comprises goblet cells.

10.-13. (canceled)

14. The in vitro method according to claim 1, which further comprises dissociating cells that are comprised in the conjunctiva organoid and/or the limbal organoid and subsequently culturing one or more of the dissociated cells in an air-liquid interface culture, wherein the method comprises the steps of:
(a) providing a container comprising a first chamber and a second chamber, said first and second chambers separated by a porous membrane;
(b) seeding of cells dissociated from the conjunctiva organoid on one side of the porous membrane, preferably in the first chamber:
(c) providing culture medium to the first and/or to the second chamber such that the seeded cells on the porous membrane are submerged in the culture medium
(d) allowing the seeded cells to expand on the porous membrane for a period of time sufficient to obtain a substantially confluent layer of cells; and
(e) removing the culture medium from the chamber that includes the layer of confluent cells- and allowing the cells to be in contact with air for period of time sufficient to obtain a conjunctiva cell culture comprising multiple layers of cells, wherein the cell layers proximal to the porous membrane comprise basal keratinocytes that express KRT19 and TP63 and wherein the cell layers distal to the porous membrane comprise apical keratinocytes that express MUC1, and goblet cells, in particular that express MUC5.

15. (canceled)

16. An isolated conjunctiva organoid, in particular a human conjunctiva organoid, comprising keratinocytes that express KRT19, and one or more of TP63, MUC1, and AQP5, and which further comprises Goblet cells, preferably Goblet cells that express MUC5AC.

17.-19. (canceled)

20. The isolated conjunctiva according to claim 16, wherein the isolated conjunctiva is in the form of an conjunctiva implant.

21.-28. (canceled)

29. The isolated conjunctiva according to claim 16, wherein the isolated conjunctiva is implanted in a subject, wherein the isolated conjunctiva is in the form of an conjunctiva implant as a treatment.

30. The isolated conjunctiva according to claim 29, wherein the treatment is for the prevention and/or treatment of a disease or disorder of the human conjunctiva.
